(19) United States
(12) Patent Application Publication
(10) Pub. No.: US 2025/0121078 A1
(43) Pub. Date: Apr. 17, 2025

(54) PEPTIDE DRUG CONJUGATES AND THEIR USES

(71) Applicant: National Health Research Institutes, Miaoli County (TW)

(72) Inventors: Lun Kelvin Tsou, Miaoli County (TW); Yu-Wei Liu, Miaoli County (TW); Chiung-Tong Chen, Miaoli County (TW); Tai-Yu Chiu, Miaoli County (TW); Chuan Shih, Westfield, IN (US); Jang-Yang Chang, Taipei (TW)

(21) Appl. No.: 18/906,730

(22) Filed: Oct. 4, 2024

Related U.S. Application Data
(60) Provisional application No. 63/542,861, filed on Oct. 6, 2023.

(51) Int. Cl. A61K 47/64; A61K 47/54; A61P 35/00

(52) U.S. Cl. CPC A61K 47/64; A61K 47/545; A61P 35/00

(57) ABSTRACT

Disclosed are compounds of formula (I):

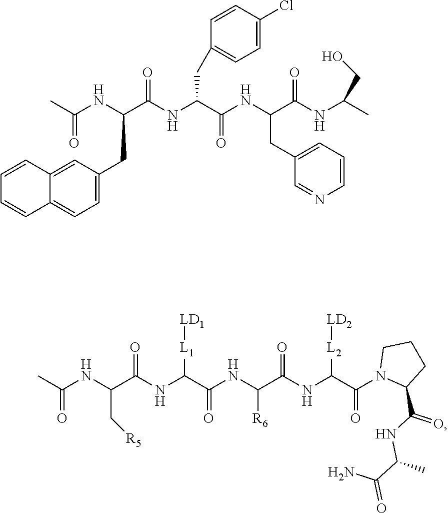

in which L₁, L₂, LD₁, LD₂, R₅, and R₆ are defined. Also provided are pharmaceutical compositions containing such a compound a method of treating cancer using the compound.

### CROSS-REFERENCE TO RELATED APPLICATION

This application claims the benefit of priority to U.S. Provisional Application No. 63/542,861 filed Oct. 6, 2023, the entire content of which is incorporated by reference.

### BACKGROUND

Luteinizing hormone-releasing hormone (LHRH) is a useful ligand targeting LHRH receptors (LHRH-R) that are overexpressed in the plasma membrane of several types of cancer cells and are not expressed detectably in many normal organs.

The incidence of LHRH-R expression in various cancers was reported to be high. LHRH-R are expressed in about 86% of prostate cancer, 80% of human endometrial and ovarian cancers, 80% of renal cancer, 50% of breast cancers, and 32-50% of pancreatic cancers. See Li et al., Mini Rev Med Chem 17 (3), 258-267 (2017).

As such, LHRH-R receptors are of high interest in the targeted cancer therapy approach as they provide the desired properties to allow selective tumor targeting. See Reubi, 2003 and Vhora et al., 2014.

A major challenge in the development of novel and highly effective LHRH drug conjugates is the selective drug-delivery to the tumor site while healthy tissue is spared. One strategy is to covalently conjugate chemotherapeutic agents to antibodies that facilitate the recognition of specific tumor antigens. However, antibody-drug conjugates are limited by poor in vitro and in vivo stability, high antigenicity, complicated conjugation chemistry, relative high manufacturing cost, and marginal solid tumor penetration. Further, an effective LHRH-drug conjugate should be stable enough to transport a therapeutic agent to cancer cells and release it at a desired moment for killing the cancer cells.

There is a need to develop new LHRH-drug conjugates for delivering therapeutic agents efficiently to cancer cells without the above-described drawbacks.

### SUMMARY

To address the above need, certain LHRH compounds have been designed that surprisingly conjugate with a drug moiety for cancer treatment.

Accordingly, one aspect of this invention relates to compounds of formula (I):

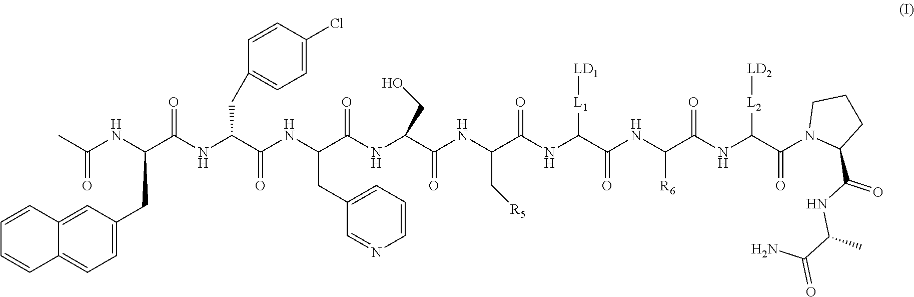

In formula (I) above,
each of L₁ and L₂, independently, is C₁-C₆ alkyl, aryl, or aralkyl;
LD₁ is —N(R₁)(R₂) or a first linker-drug moiety represented by -LK1-DG1, in which R₁ is H, R₂ is H or substituted carbonyl, or R₁ and R₂, together with the nitrogen atom they attached to, form —N₃, substituted or unsubstituted guanidino, 5- to 14-membered heteroaryl, or 5- to 14-membered heterocycloalkyl; LK1 is a first linker; and DG1 is a first drug moiety;
LD₂ is —N(R₃)(R₄) or a second linker-drug moiety represented by -LK2-DG2, in which R₃ is H, R₄ is substituted carbonyl, or R₃ and R₄, together with the nitrogen atom they attached to, form substituted or unsubstituted guanidino, 5- to 14-membered heteroaryl, or 5- to 14-membered heterocycloalkyl; LK2 is a second linker; and DG2 is a second drug moiety;
R₅ is aryl, C₁-C₆ alkyl, or a third linker-drug moiety represented by -LK3-DG3, in which LK3 is a third linker and DG3 is a third drug moiety; and
R₆ is C₁-C₆ alkyl.

Compounds of formula (I) can have one or more of the features below, in any combinations:
(i) each of moieties LD₁ and LD₂, independently, is —NH₂, —N₃, —NHC(O)NH₂, —NHC(═NH)NH₂, or —N═C(NHCH₂CH₃)₂;
(ii) LD₂ is —NHC(O)NH₂, —NHC(═NH)NH₂, or —N═C(NHCH₂CH₃)₂;
(iii) one of LD₁ and LD₂ is —NHC(O)NH₂, —NHC(═NH)NH₂, or —N═C(NHCH₂CH₃)₂, and the other is the first or second linker-drug moiety.
(iv) one of LD₁, LD₂, and R₅ is selected from the group consisting of

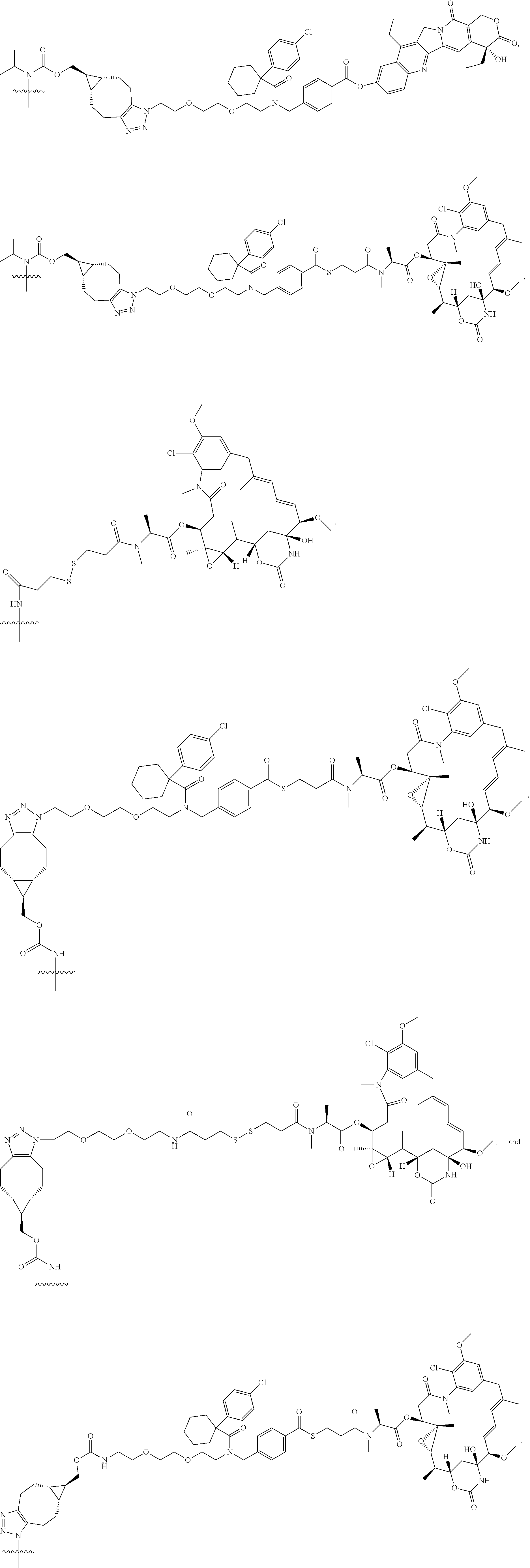

(v) R₅ is substituted phenyl or substituted C₁-C₆ alkyl;

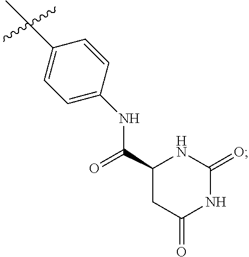 [SMILES: *c1ccc(NC(=O)[C@@H]2CC(=O)NC(=O)N2)cc1]

(vi) R₅ is aminopropyl, 4-hydroxyphenyl, or
(vii) R₆ is isobutyl or aminobutyl;
(viii) L₁ is ethylene, propylene, or phenylene; and
(ix) n is 3 or 4.

A subset of the compounds of formula (I) includes the compounds of formula (II) or (III) below:

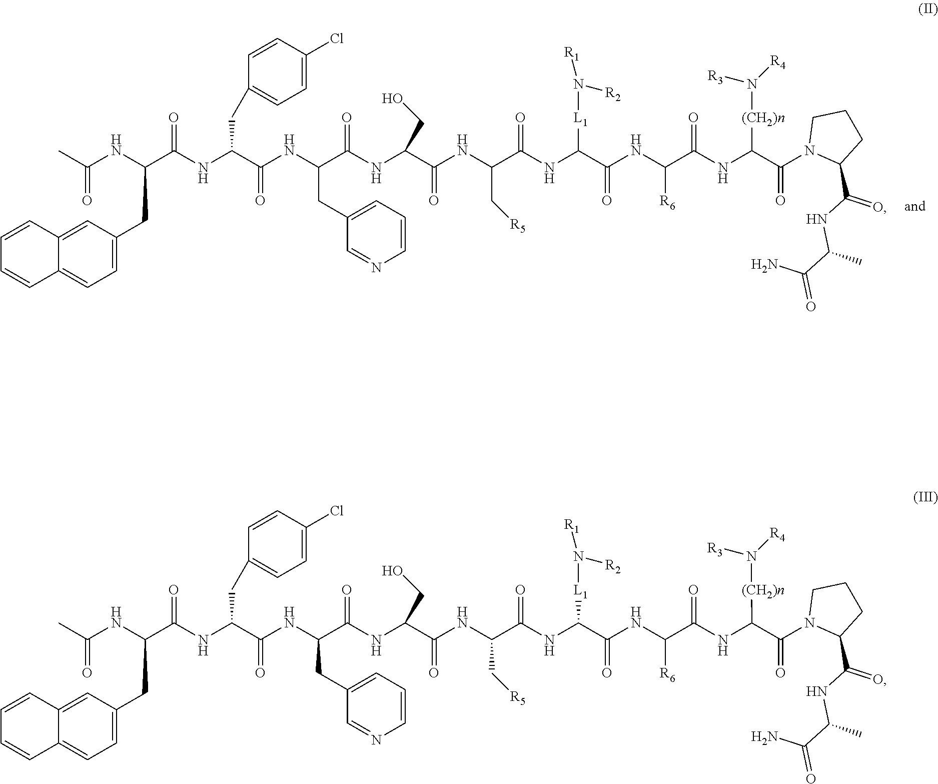

in which n is 1-6, and each of R₁-R₆ and L₁ is defined above.

Another aspect of the invention relates to a pharmaceutical composition comprising a compound of any one of the compounds described above and a pharmaceutically acceptable carrier.

Also included in the scope of the invention is a method of treating cancer comprising administering an effective amount of any compound described above to a patient in need thereof.

Exemplary compounds of formula (I) include Compounds 1-32, the structures of which are shown below.

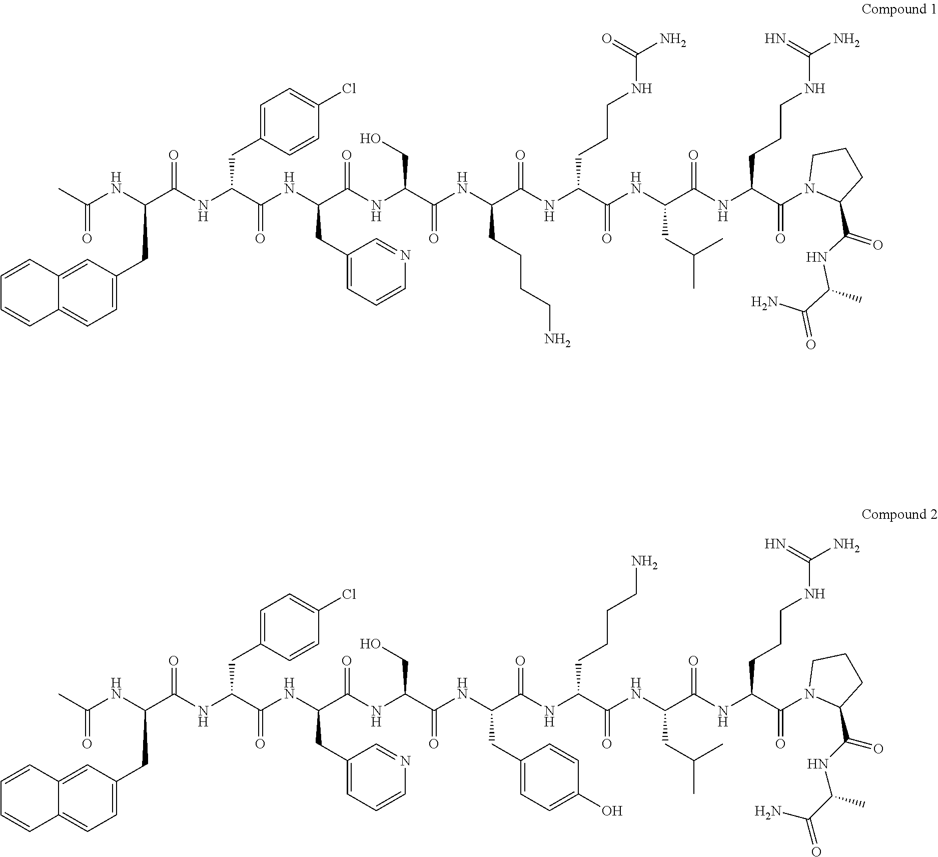 [SMILES: CC(=O)N[C@H](Cc1ccc2ccccc2c1)C(=O)N[C@H](Cc1ccc(Cl)cc1)C(=O)N[C@H](Cc1cccnc1)C(=O)N[C@@H](CO)C(=O)N[C@H](CCCCN)C(=O)N[C@H](CCCNC(N)=O)C(=O)N[C@@H](CC(C)C)C(=O)N[C@@H](CCCNC(=N)N)C(=O)N1CCC[C@H]1C(=O)N[C@H](C)C(N)=O ; CC(=O)N[C@H](Cc1ccc2ccccc2c1)C(=O)N[C@H](Cc1ccc(Cl)cc1)C(=O)N[C@H](Cc1cccnc1)C(=O)N[C@@H](CO)C(=O)N[C@@H](Cc1ccc(O)cc1)C(=O)N[C@H](CCCCN)C(=O)N[C@@H](CC(C)C)C(=O)N[C@@H](CCCNC(=N)N)C(=O)N1CCC[C@H]1C(=O)N[C@H](C)C(N)=O]

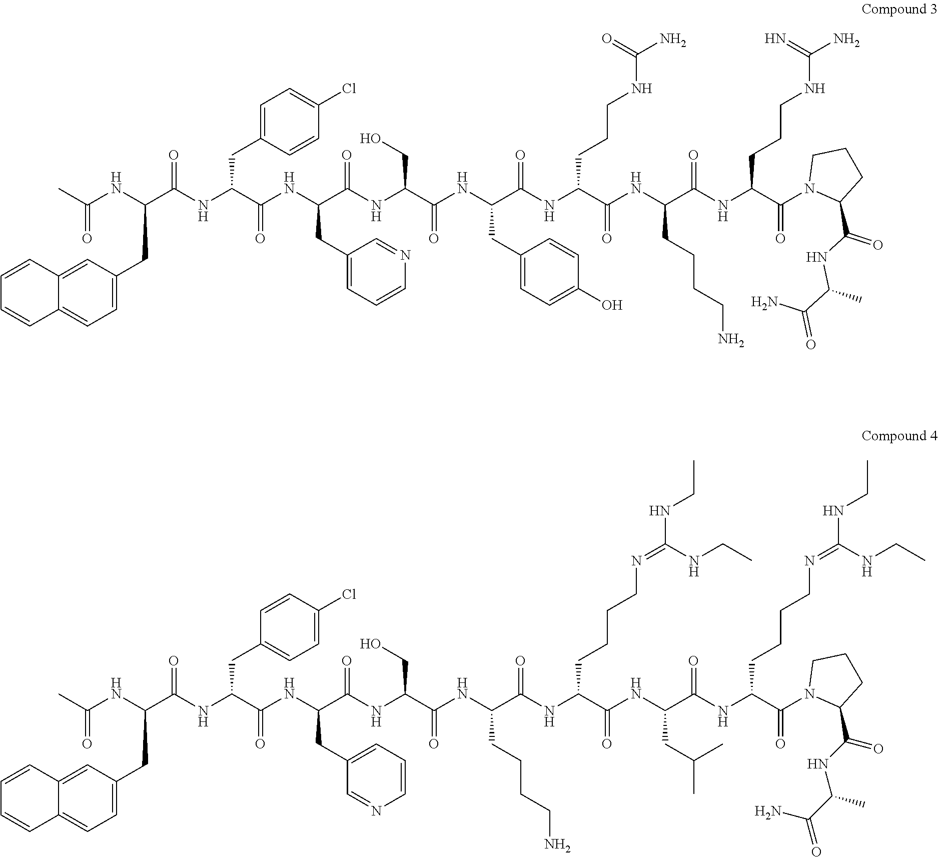 [SMILES: CC(=O)N[C@H](Cc1ccc2ccccc2c1)C(=O)N[C@H](Cc1ccc(Cl)cc1)C(=O)N[C@H](Cc1cccnc1)C(=O)N[C@@H](CO)C(=O)N[C@@H](Cc1ccc(O)cc1)C(=O)N[C@H](CCCNC(N)=O)C(=O)N[C@H](CCCCN)C(=O)N[C@@H](CCCNC(=N)N)C(=O)N1CCC[C@H]1C(=O)N[C@H](C)C(N)=O ; CCNC(=NCCCC[C@@H](NC(=O)[C@H](CCCCN)NC(=O)[C@H](CO)NC(=O)[C@@H](Cc1cccnc1)NC(=O)[C@@H](Cc1ccc(Cl)cc1)NC(=O)[C@@H](Cc1ccc2ccccc2c1)NC(C)=O)C(=O)N[C@@H](CC(C)C)C(=O)N[C@H](CCCCN=C(NCC)NCC)C(=O)N1CCC[C@H]1C(=O)N[C@H](C)C(N)=O)NCC]

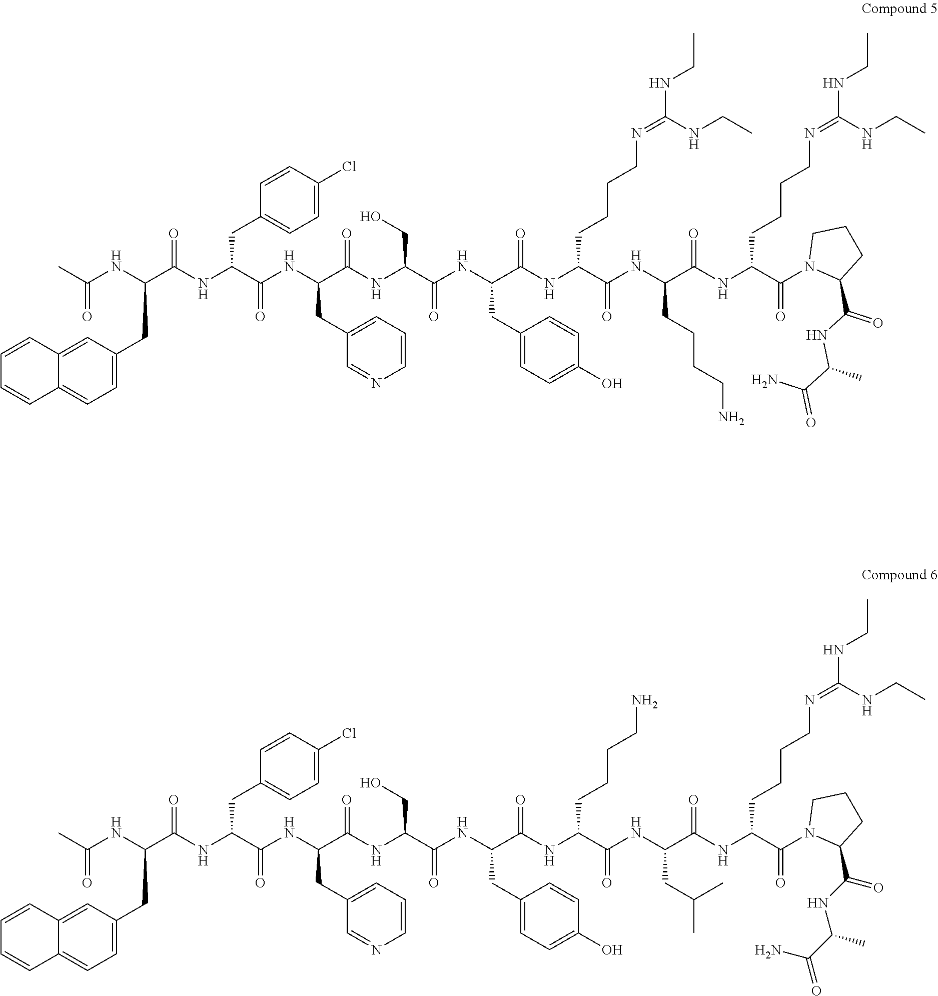 [SMILES: CCNC(=NCCCC[C@@H](NC(=O)[C@H](Cc1ccc(O)cc1)NC(=O)[C@H](CO)NC(=O)[C@@H](Cc1cccnc1)NC(=O)[C@@H](Cc1ccc(Cl)cc1)NC(=O)[C@@H](Cc1ccc2ccccc2c1)NC(C)=O)C(=O)N[C@H](CCCCN)C(=O)N[C@H](CCCCN=C(NCC)NCC)C(=O)N1CCC[C@H]1C(=O)N[C@H](C)C(N)=O)NCC ; CCNC(=NCCCC[C@@H](NC(=O)[C@H](CC(C)C)NC(=O)[C@@H](CCCCN)NC(=O)[C@H](Cc1ccc(O)cc1)NC(=O)[C@H](CO)NC(=O)[C@@H](Cc1cccnc1)NC(=O)[C@@H](Cc1ccc(Cl)cc1)NC(=O)[C@@H](Cc1ccc2ccccc2c1)NC(C)=O)C(=O)N1CCC[C@H]1C(=O)N[C@H](C)C(N)=O)NCC]

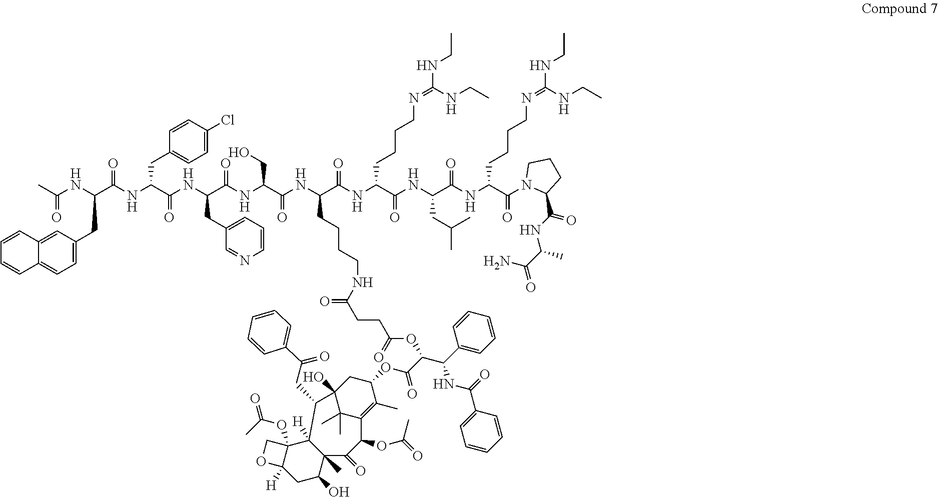 [SMILES: CCNC(=NCCCC[C@@H](NC(=O)[C@@H](CCCCNC(=O)CCC(=O)O[C@@H](C(=O)O[C@H]1C[C@@]2(O)[C@@H](CC(=O)c3ccccc3)[C@@H]3[C@]4(OC(C)=O)CO[C@@H]4C[C@H](O)[C@@]3(C)C(=O)[C@H](OC(C)=O)C(=C1C)C2(C)C)[C@@H](NC(=O)c1ccccc1)c1ccccc1)NC(=O)[C@H](CO)NC(=O)[C@@H](Cc1cccnc1)NC(=O)[C@@H](Cc1ccc(Cl)cc1)NC(=O)[C@@H](Cc1ccc2ccccc2c1)NC(C)=O)C(=O)N[C@@H](CC(C)C)C(=O)N[C@H](CCCCN=C(NCC)NCC)C(=O)N1CCC[C@H]1C(=O)N[C@H](C)C(N)=O)NCC]

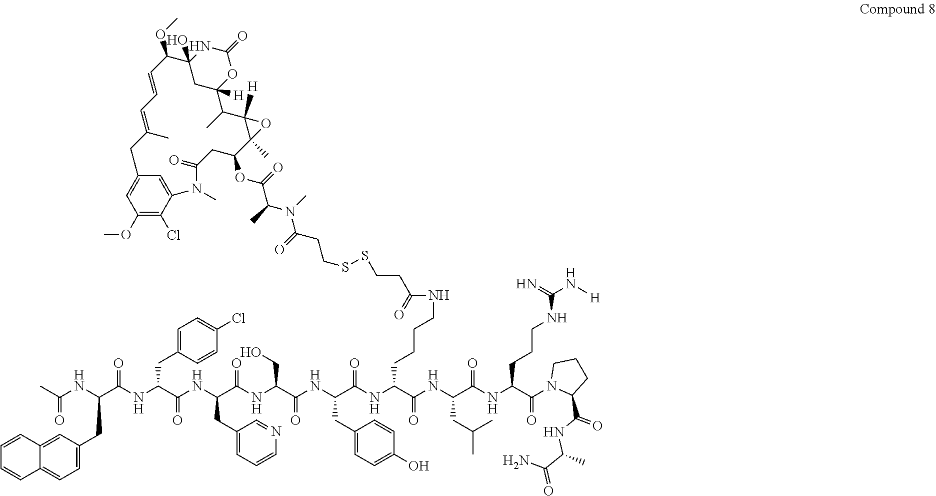

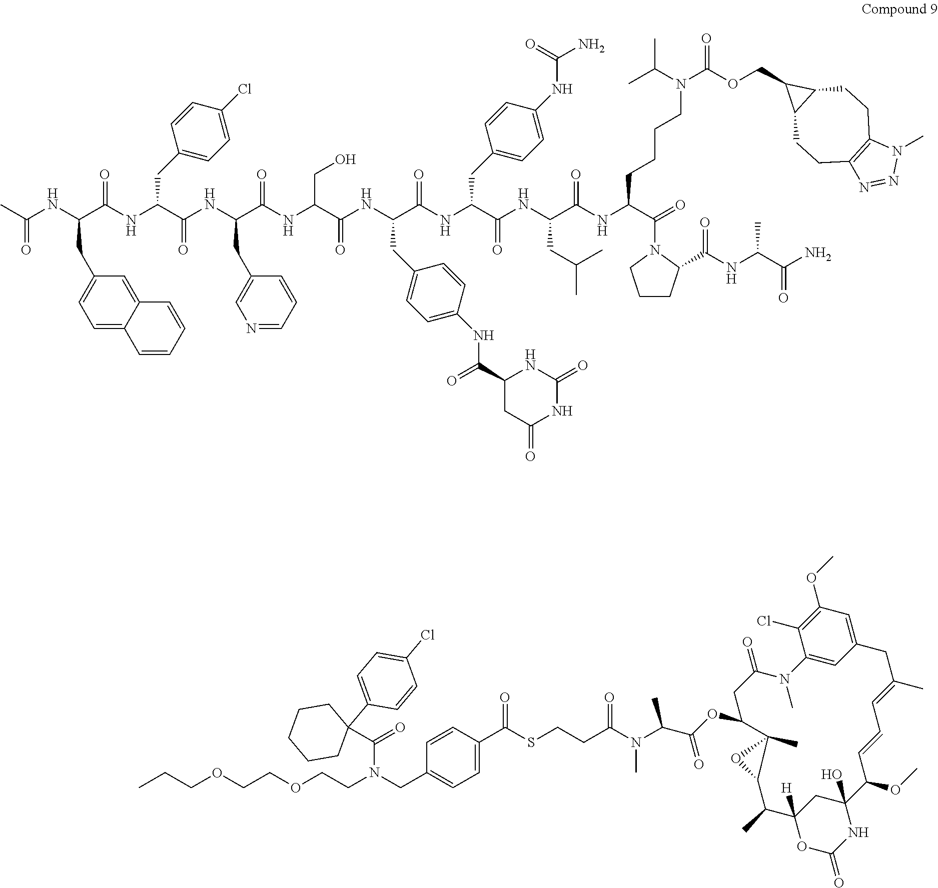

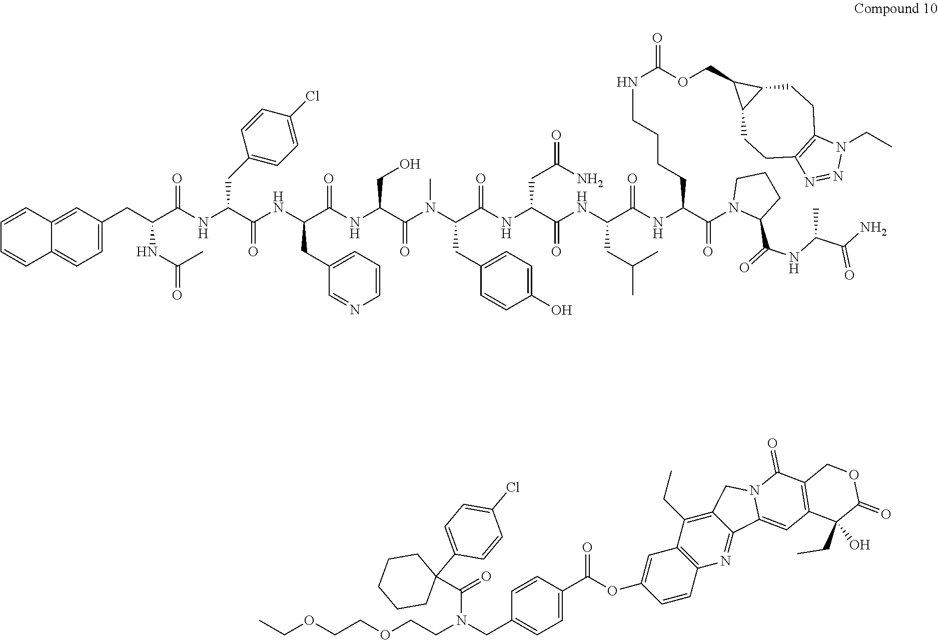

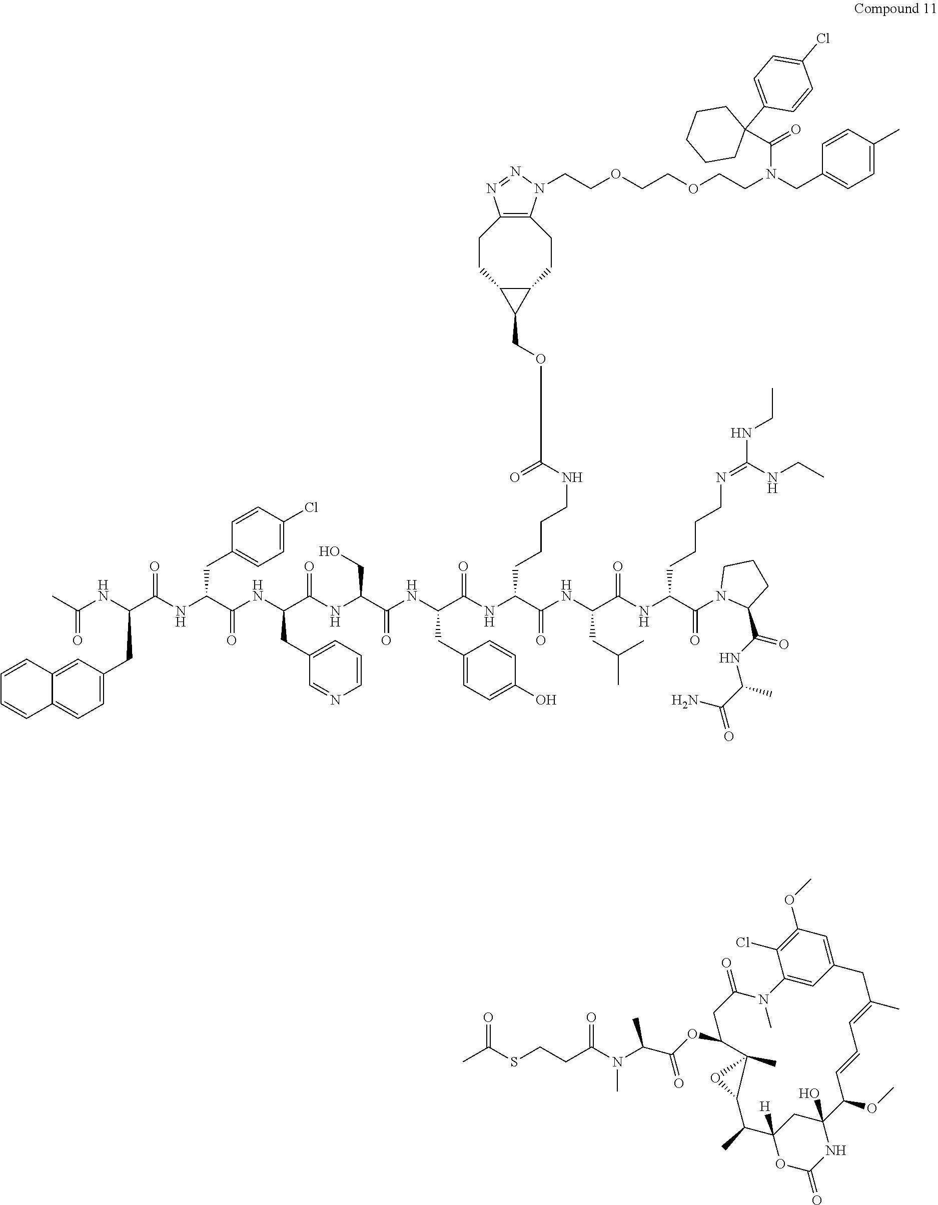

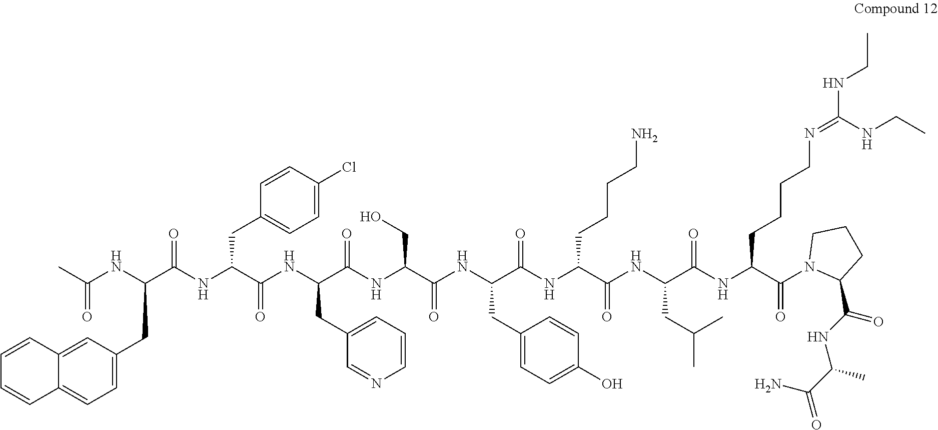 [SMILES: CCNC(=NCCCC[C@H](NC(=O)[C@H](CC(C)C)NC(=O)[C@@H](CCCCN)NC(=O)[C@H](Cc1ccc(O)cc1)NC(=O)[C@H](CO)NC(=O)[C@@H](Cc1cccnc1)NC(=O)[C@@H](Cc1ccc(Cl)cc1)NC(=O)[C@@H](Cc1ccc2ccccc2c1)NC(C)=O)C(=O)N1CCC[C@H]1C(=O)N[C@H](C)C(N)=O)NCC]

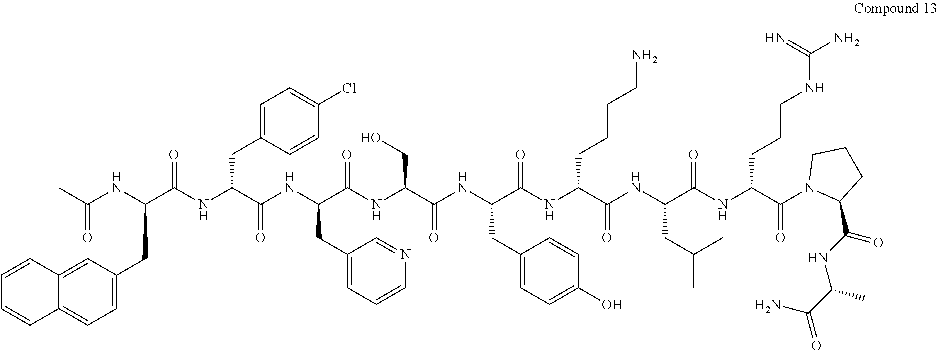 [SMILES: CC(=O)N[C@H](Cc1ccc2ccccc2c1)C(=O)N[C@H](Cc1ccc(Cl)cc1)C(=O)N[C@H](Cc1cccnc1)C(=O)N[C@@H](CO)C(=O)N[C@@H](Cc1ccc(O)cc1)C(=O)N[C@H](CCCCN)C(=O)N[C@@H](CC(C)C)C(=O)N[C@H](CCCNC(=N)N)C(=O)N1CCC[C@H]1C(=O)N[C@H](C)C(N)=O]

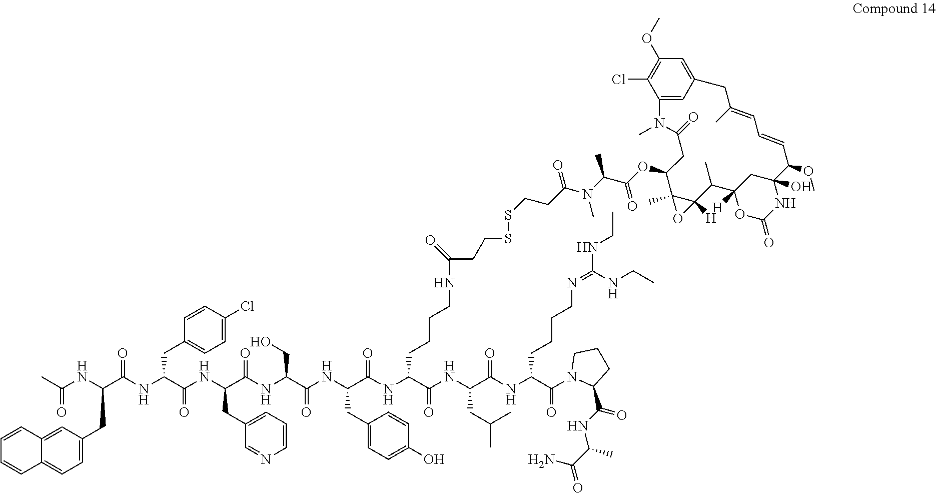 [SMILES: CCNC(=NCCCC[C@@H](NC(=O)[C@H](CC(C)C)NC(=O)[C@@H](CCCCNC(=O)CCSSCCC(=O)N(C)[C@@H](C)C(=O)O[C@H]1CC(=O)N(C)c2cc(cc(OC)c2Cl)C/C(C)=C/C=C/[C@@H](OC)[C@@]2(O)C[C@H](OC(=O)N2)C(C)[C@@H]2O[C@@]12C)NC(=O)[C@H](Cc1ccc(O)cc1)NC(=O)[C@H](CO)NC(=O)[C@@H](Cc1cccnc1)NC(=O)[C@@H](Cc1ccc(Cl)cc1)NC(=O)[C@@H](Cc1ccc2ccccc2c1)NC(C)=O)C(=O)N1CCC[C@H]1C(=O)N[C@H](C)C(N)=O)NCC]

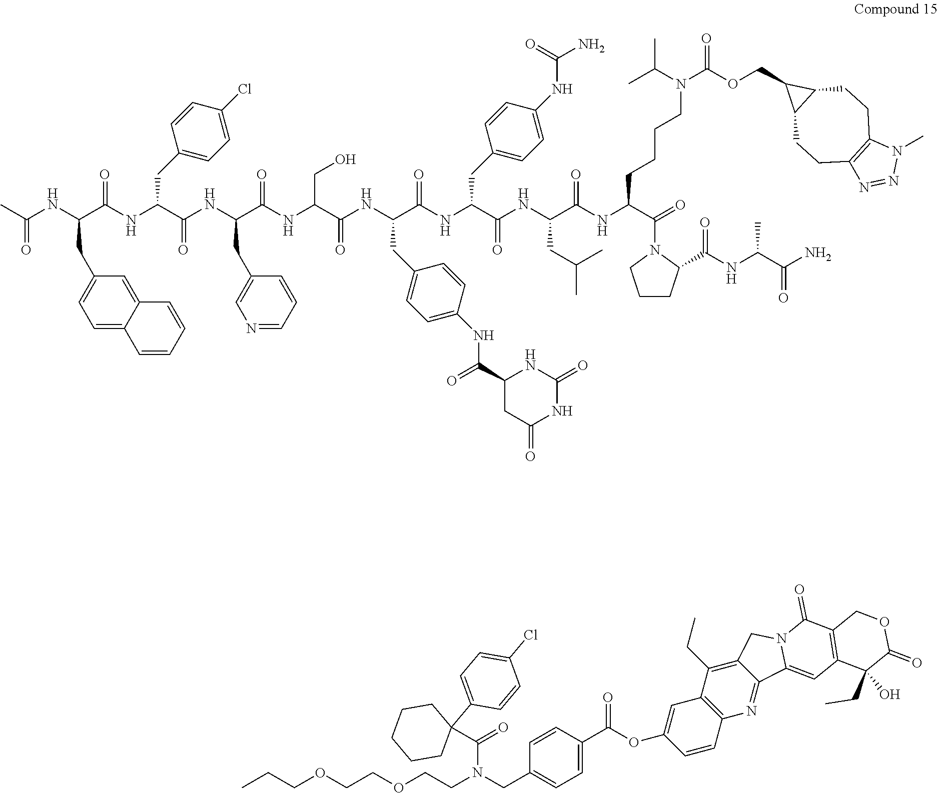

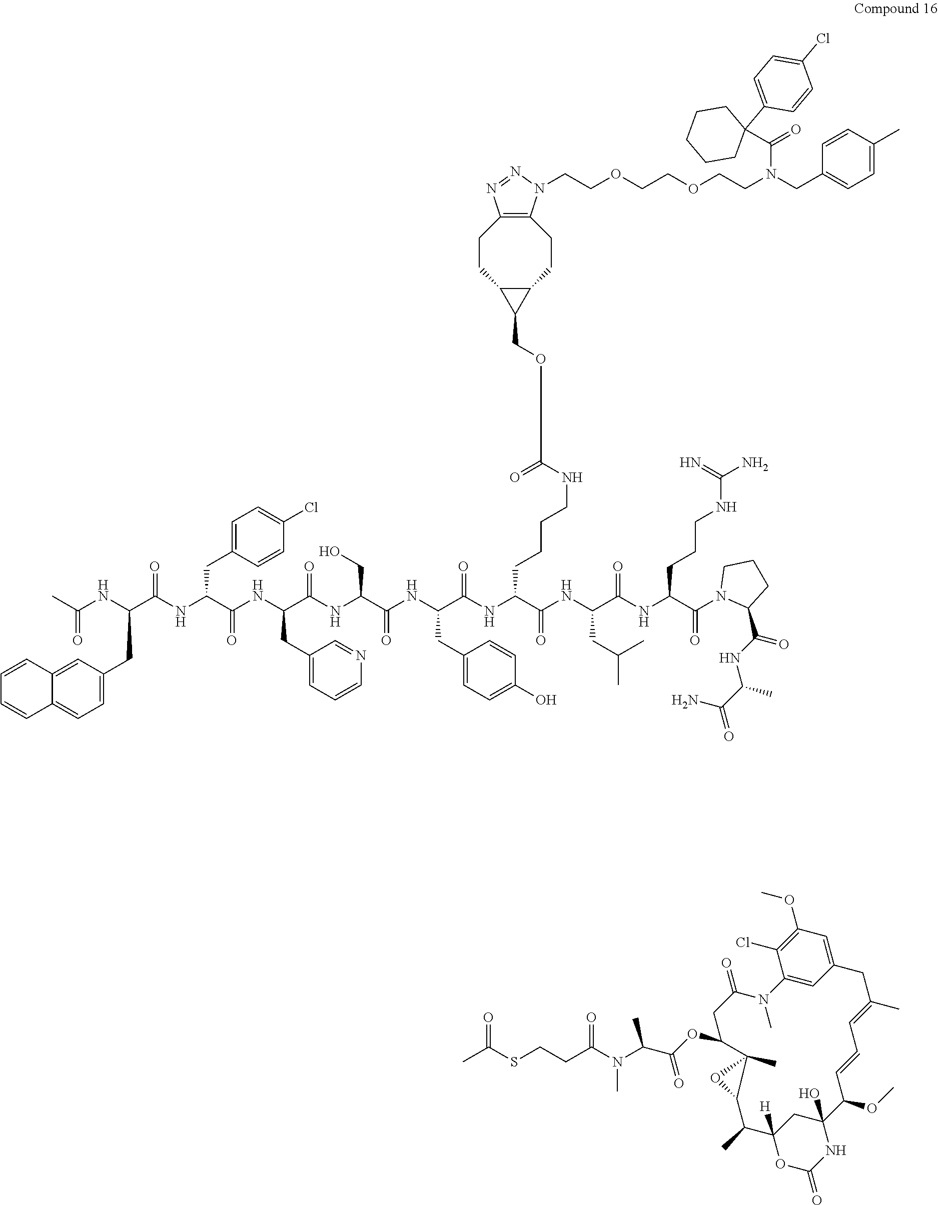

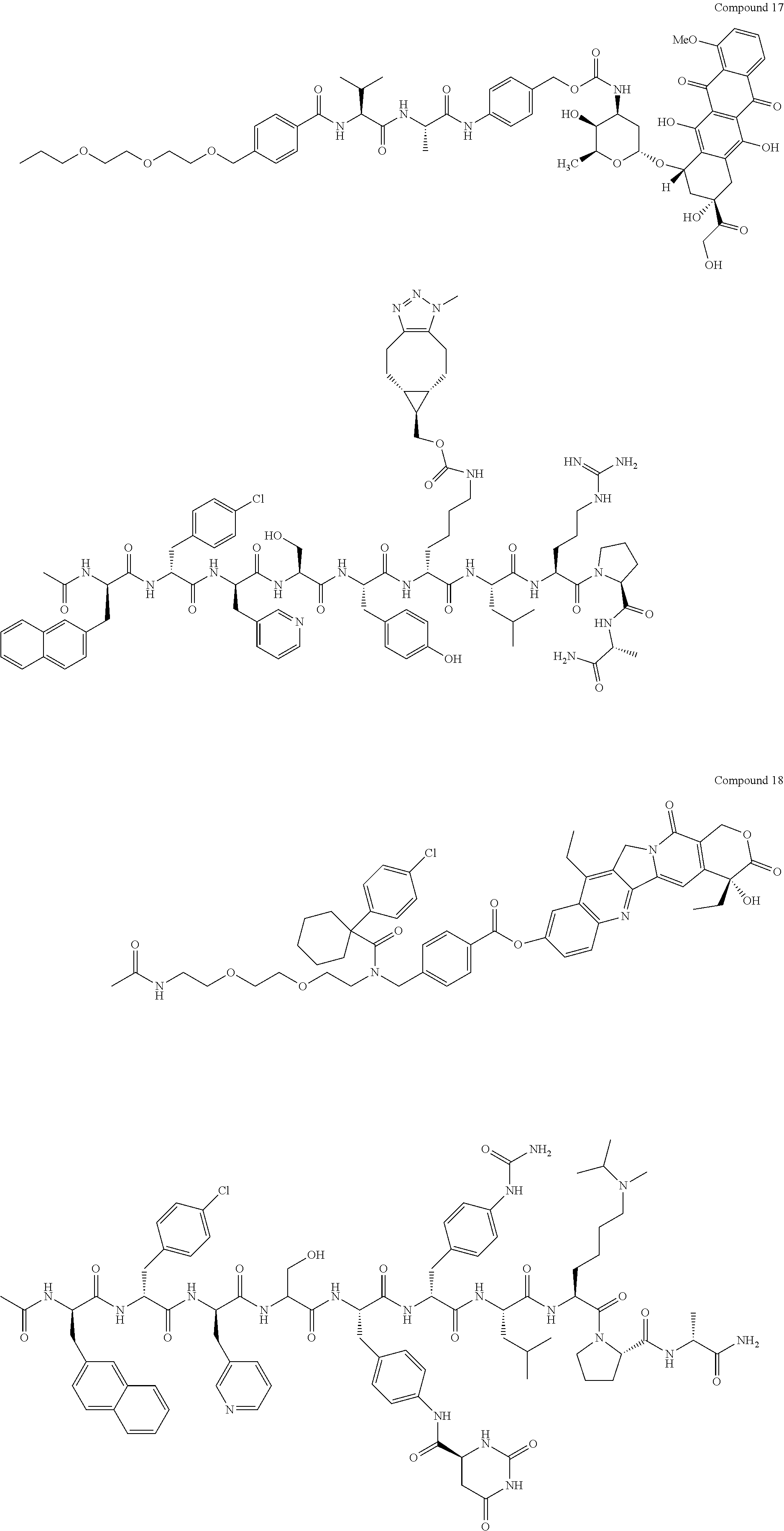

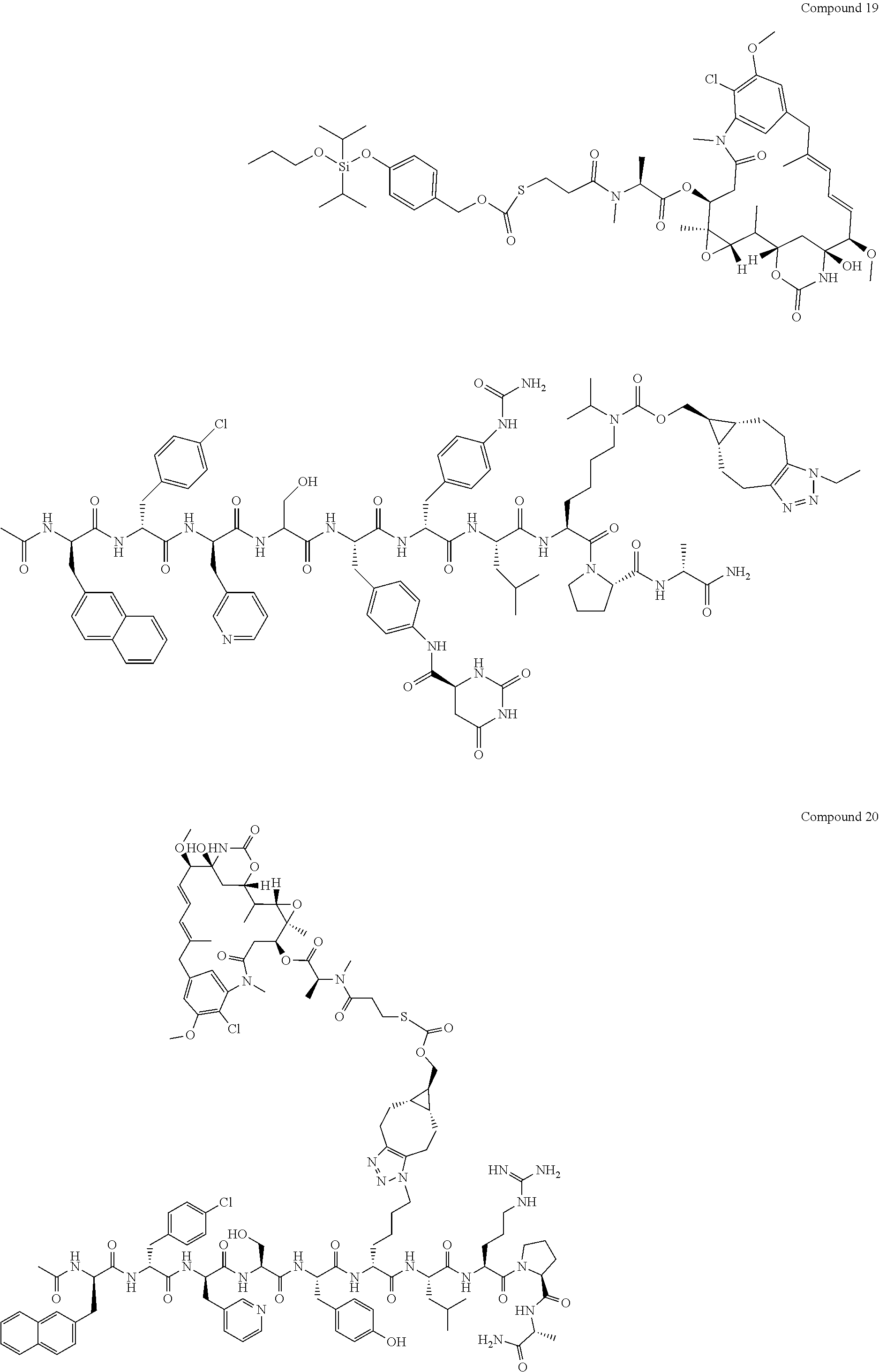

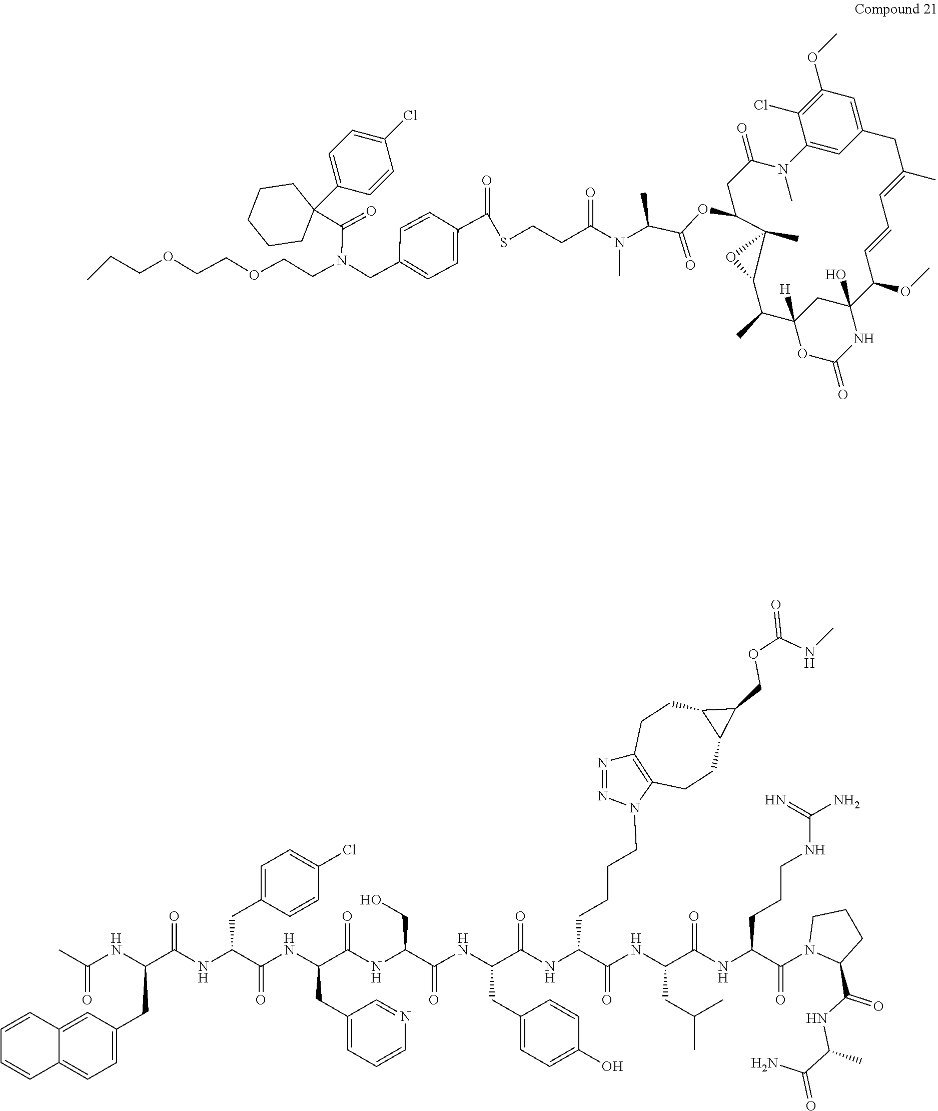 [SMILES: CCCOCCOCCN(Cc1ccc(C(=O)SCCC(=O)N(C)[C@@H](C)C(=O)O[C@H]2CC(=O)N(C)c3cc(cc(OC)c3Cl)C/C(C)=C/C=C/[C@@H](OC)[C@@]3(O)C[C@H](OC(=O)N3)[C@@H](C)[C@@H]3O[C@]23C)cc1)C(=O)C1(c2ccc(Cl)cc2)CCCCC1 ; CNC(=O)OC[C@H]1[C@@H]2CCc3c(nnn3CCCC[C@@H](NC(=O)[C@H](Cc3ccc(O)cc3)NC(=O)[C@H](CO)NC(=O)[C@@H](Cc3cccnc3)NC(=O)[C@@H](Cc3ccc(Cl)cc3)NC(=O)[C@@H](Cc3ccc4ccccc4c3)NC(C)=O)C(=O)N[C@@H](CC(C)C)C(=O)N[C@@H](CCCNC(=N)N)C(=O)N3CCC[C@H]3C(=O)N[C@H](C)C(N)=O)CC[C@@H]21]

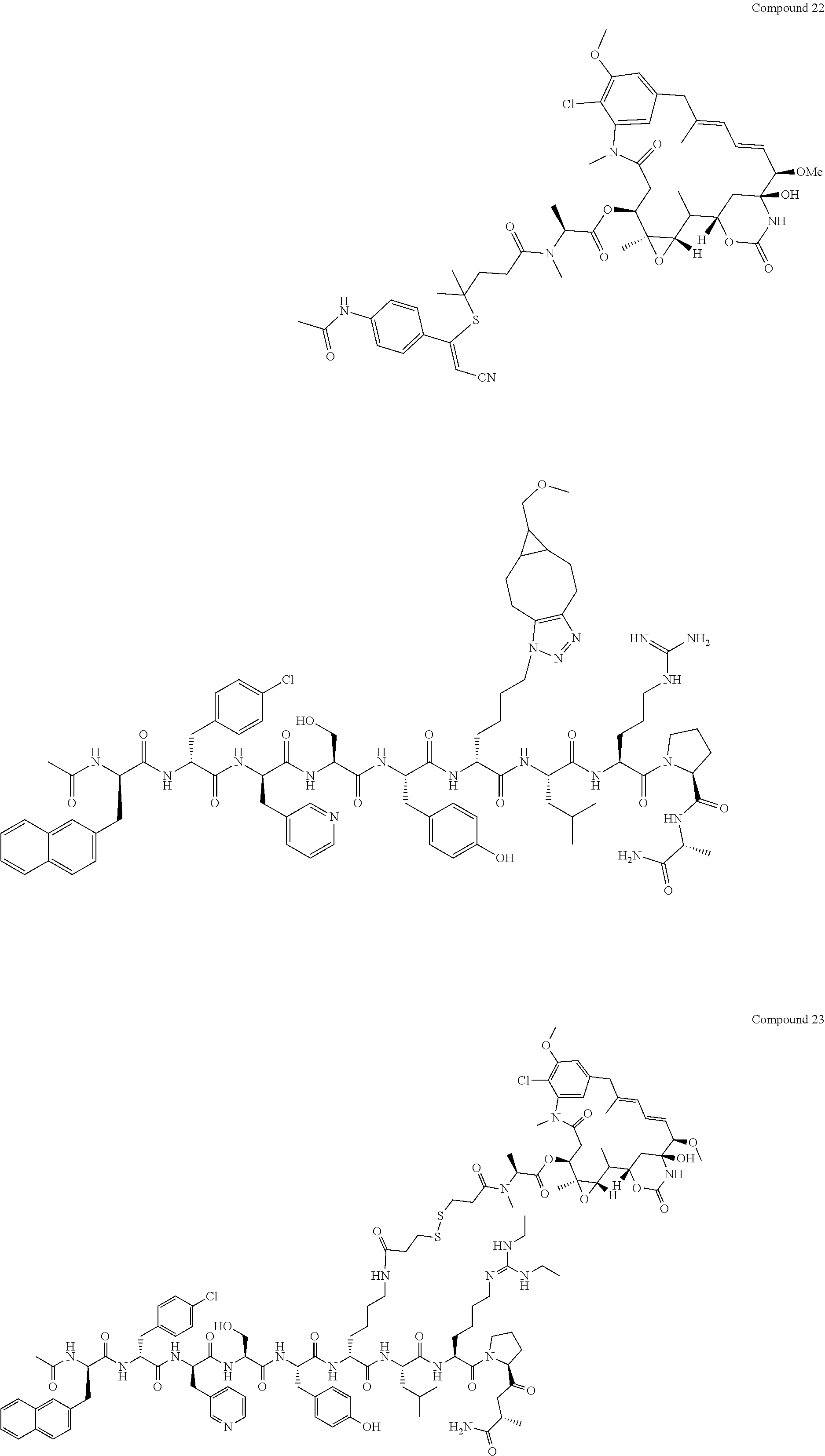

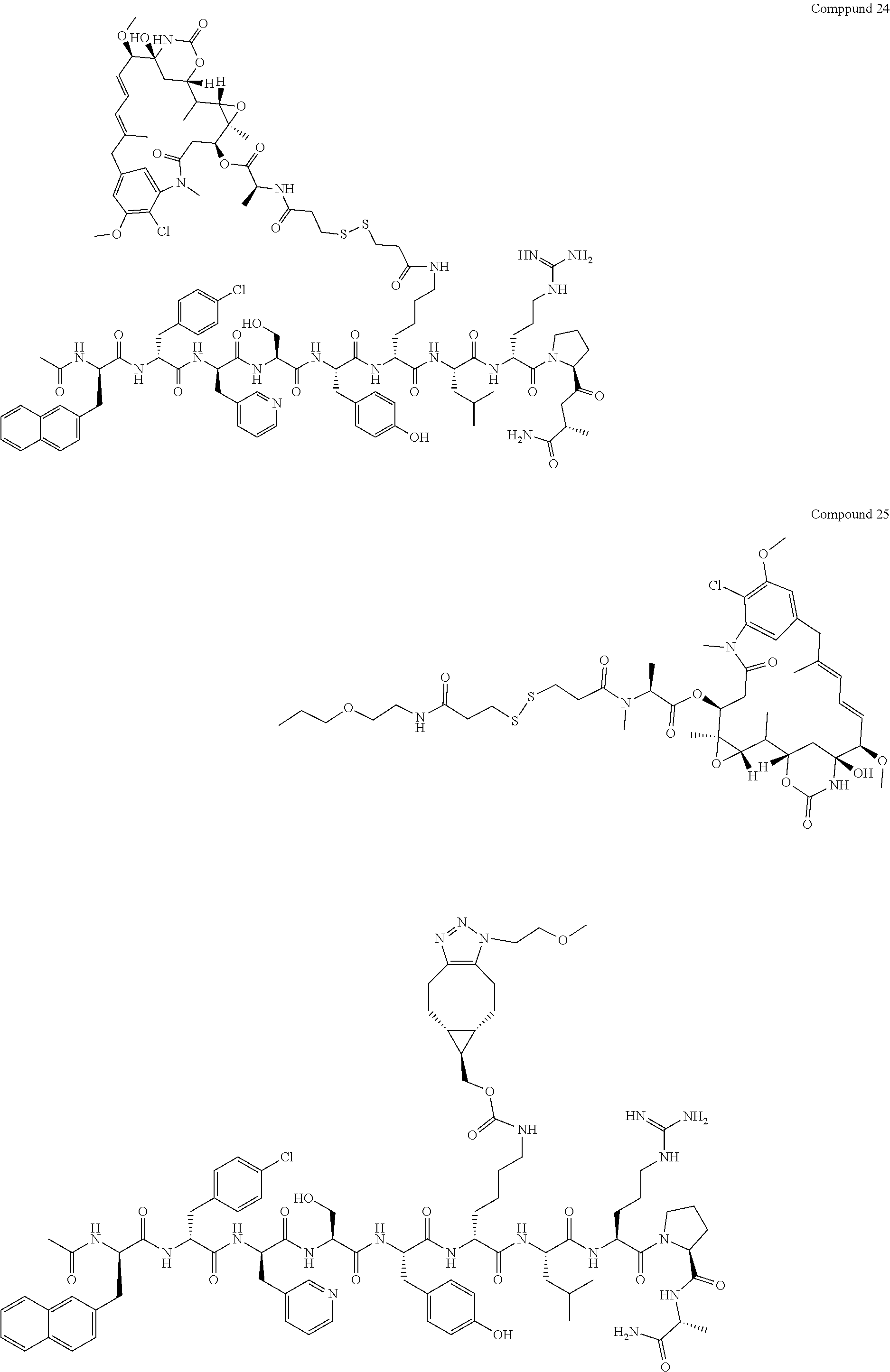

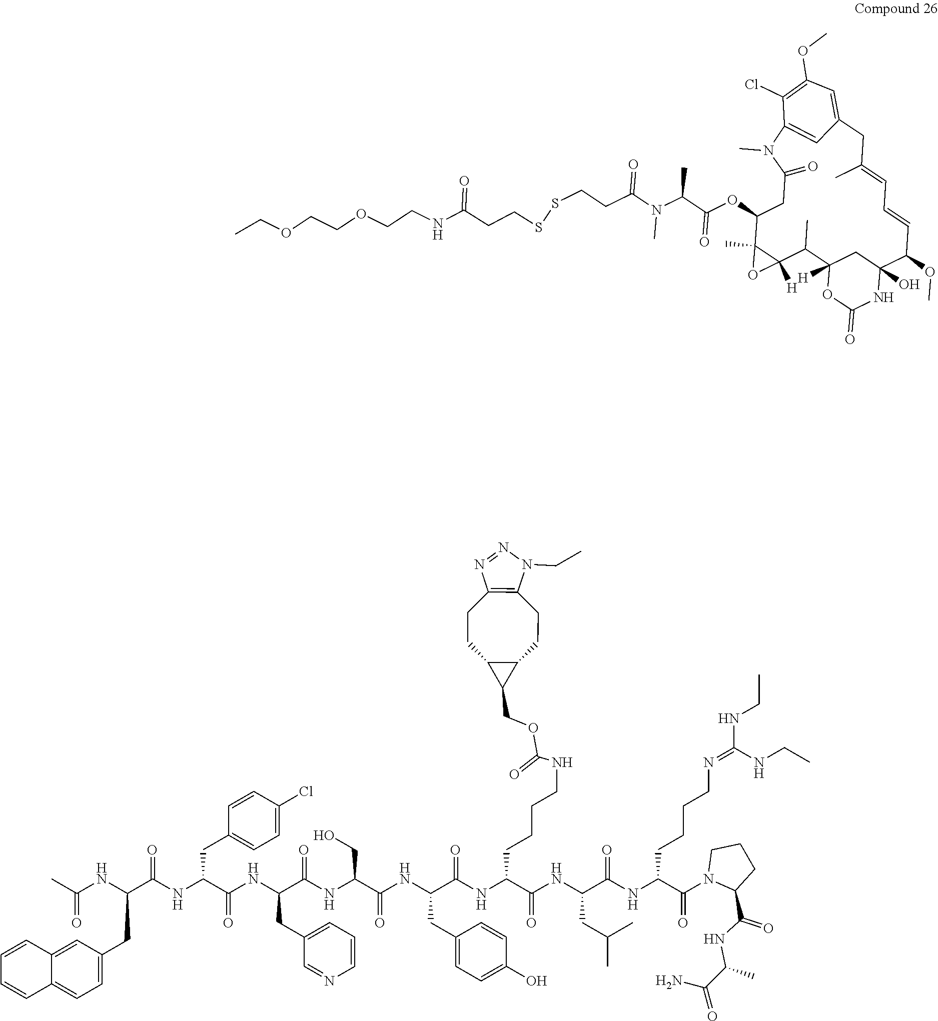

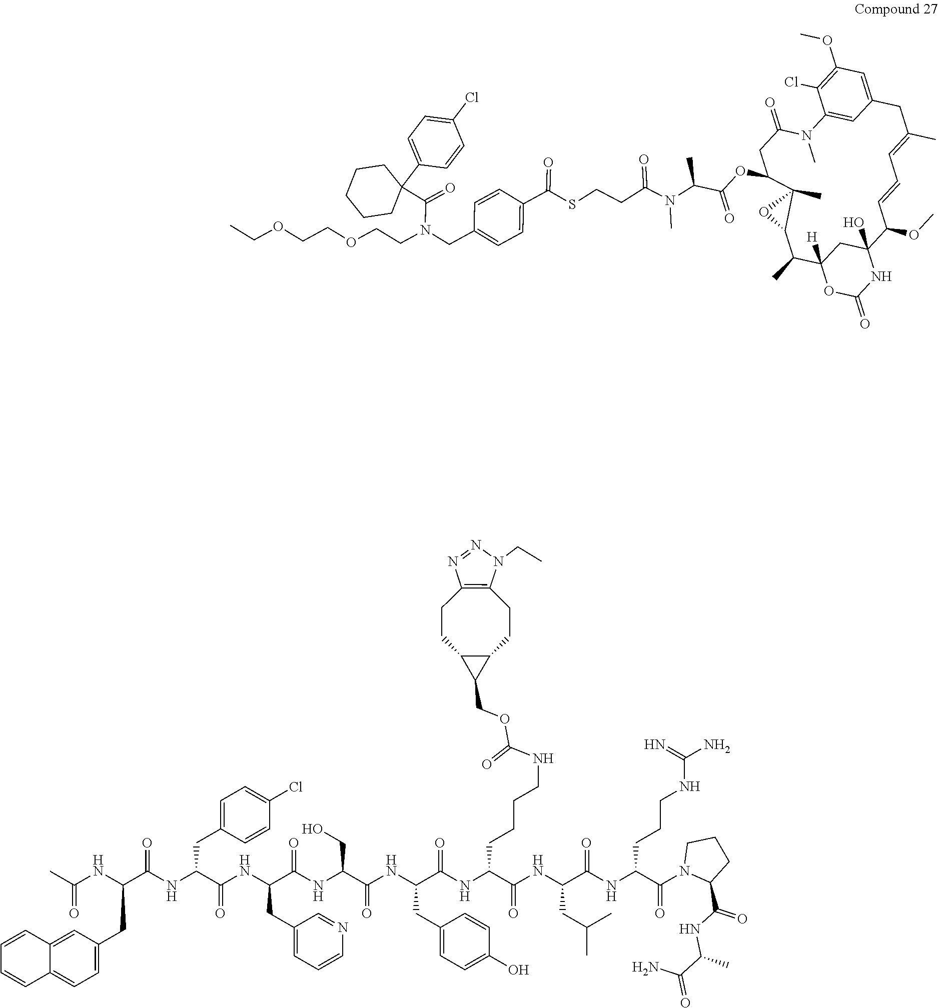 [SMILES: CCOCCOCCN(Cc1ccc(C(=O)SCCC(=O)N(C)[C@@H](C)C(=O)O[C@H]2CC(=O)N(C)c3cc(cc(OC)c3Cl)C/C(C)=C/C=C/[C@@H](OC)[C@@]3(O)C[C@H](OC(=O)N3)[C@@H](C)[C@@H]3O[C@]23C)cc1)C(=O)C1(c2ccc(Cl)cc2)CCCCC1 ; CCn1nnc2c1CC[C@H]1[C@@H](CC2)[C@@H]1COC(=O)NCCCC[C@@H](NC(=O)[C@H](Cc1ccc(O)cc1)NC(=O)[C@H](CO)NC(=O)[C@@H](Cc1cccnc1)NC(=O)[C@@H](Cc1ccc(Cl)cc1)NC(=O)[C@@H](Cc1ccc2ccccc2c1)NC(C)=O)C(=O)N[C@@H](CC(C)C)C(=O)N[C@H](CCCNC(=N)N)C(=O)N1CCC[C@H]1C(=O)N[C@H](C)C(N)=O]

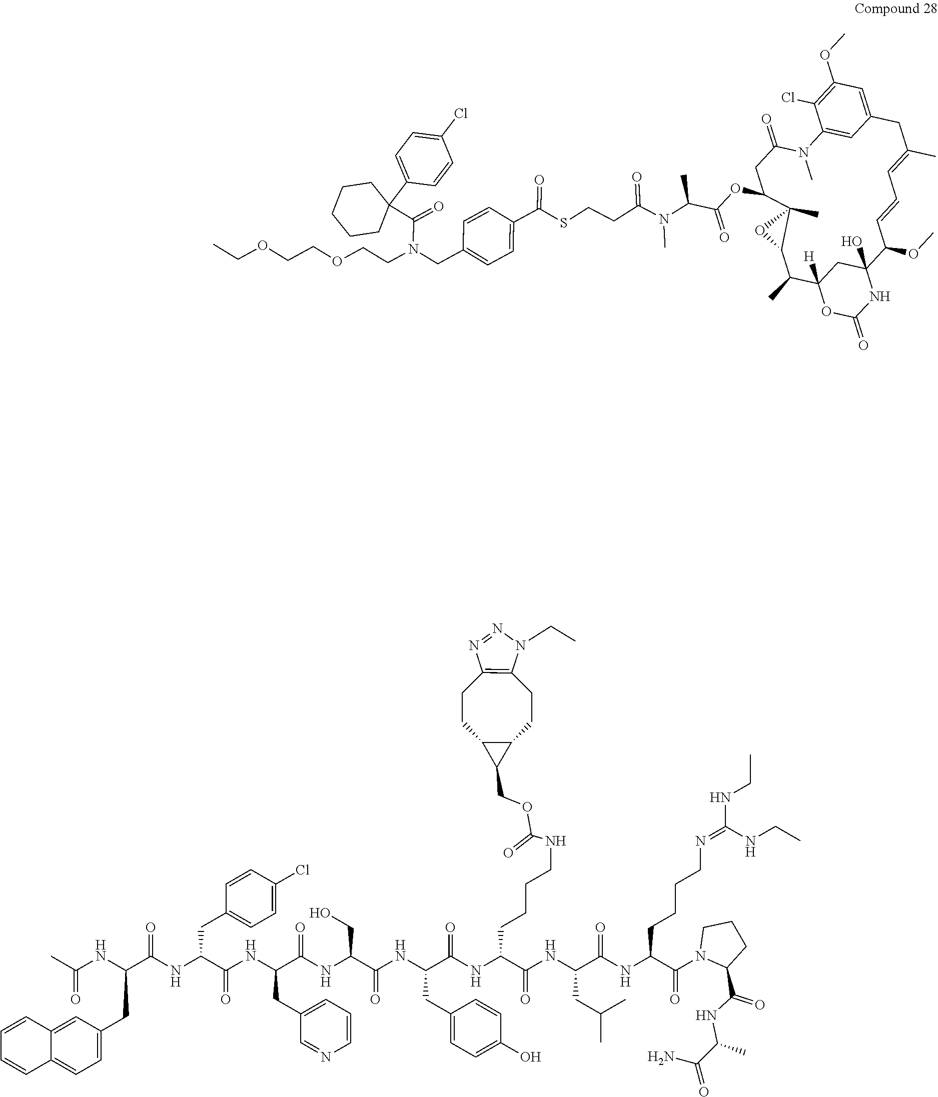 [SMILES: CCOCCOCCN(Cc1ccc(C(=O)SCCC(=O)N(C)[C@@H](C)C(=O)O[C@H]2CC(=O)N(C)c3cc(cc(OC)c3Cl)C/C(C)=C/C=C/[C@@H](OC)[C@@]3(O)C[C@H](OC(=O)N3)[C@@H](C)[C@@H]3O[C@]23C)cc1)C(=O)C1(c2ccc(Cl)cc2)CCCCC1 ; CCNC(=NCCCC[C@H](NC(=O)[C@H](CC(C)C)NC(=O)[C@@H](CCCCNC(=O)OC[C@H]1[C@@H]2CCc3nnn(CC)c3CC[C@@H]21)NC(=O)[C@H](Cc1ccc(O)cc1)NC(=O)[C@H](CO)NC(=O)[C@@H](Cc1cccnc1)NC(=O)[C@@H](Cc1ccc(Cl)cc1)NC(=O)[C@@H](Cc1ccc2ccccc2c1)NC(C)=O)C(=O)N1CCC[C@H]1C(=O)N[C@H](C)C(N)=O)NCC]

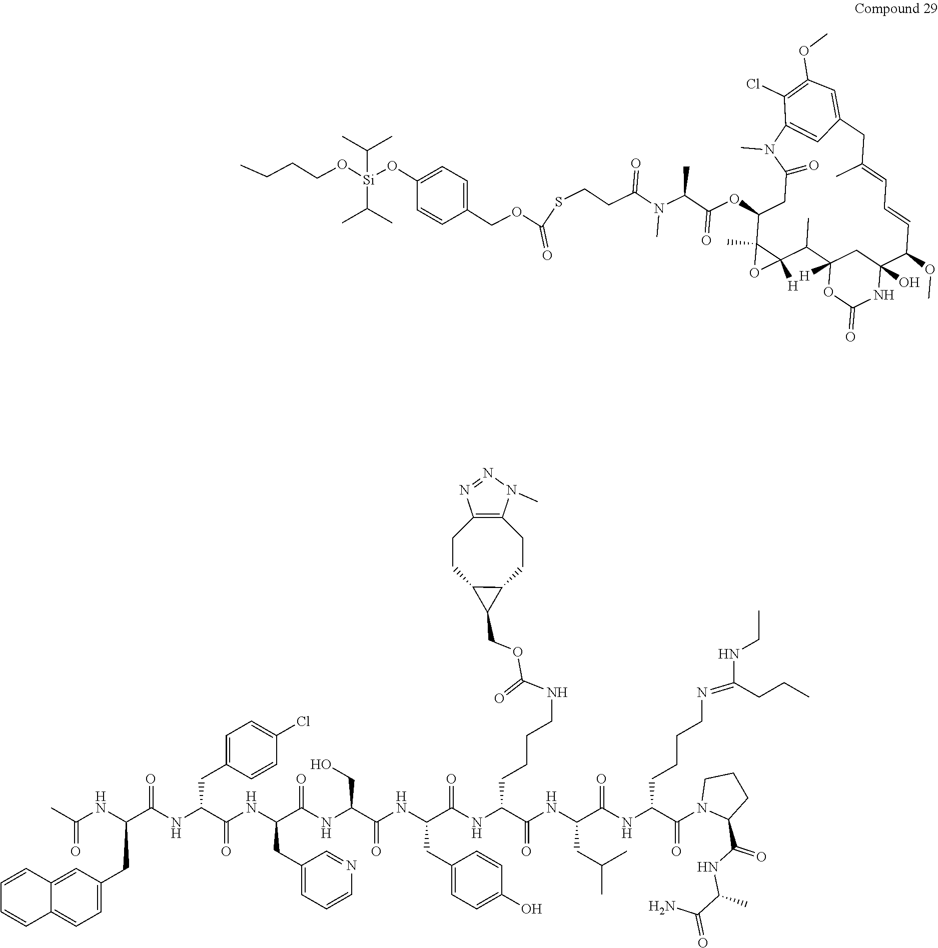 [SMILES: CCCCO[Si](Oc1ccc(COC(=O)SCCC(=O)N(C)[C@@H](C)C(=O)O[C@H]2CC(=O)N(C)c3cc(cc(OC)c3Cl)C/C(C)=C/C=C/[C@@H](OC)[C@@]3(O)C[C@H](OC(=O)N3)C(C)[C@@H]3O[C@@]23C)cc1)(C(C)C)C(C)C ; CCC/C(=N\CCCC[C@@H](NC(=O)[C@H](CC(C)C)NC(=O)[C@@H](CCCCNC(=O)OC[C@H]1[C@@H]2CCc3nnn(C)c3CC[C@@H]21)NC(=O)[C@H](Cc1ccc(O)cc1)NC(=O)[C@H](CO)NC(=O)[C@@H](Cc1cccnc1)NC(=O)[C@@H](Cc1ccc(Cl)cc1)NC(=O)[C@@H](Cc1ccc2ccccc2c1)NC(C)=O)C(=O)N1CCC[C@H]1C(=O)N[C@H](C)C(N)=O)NCC]

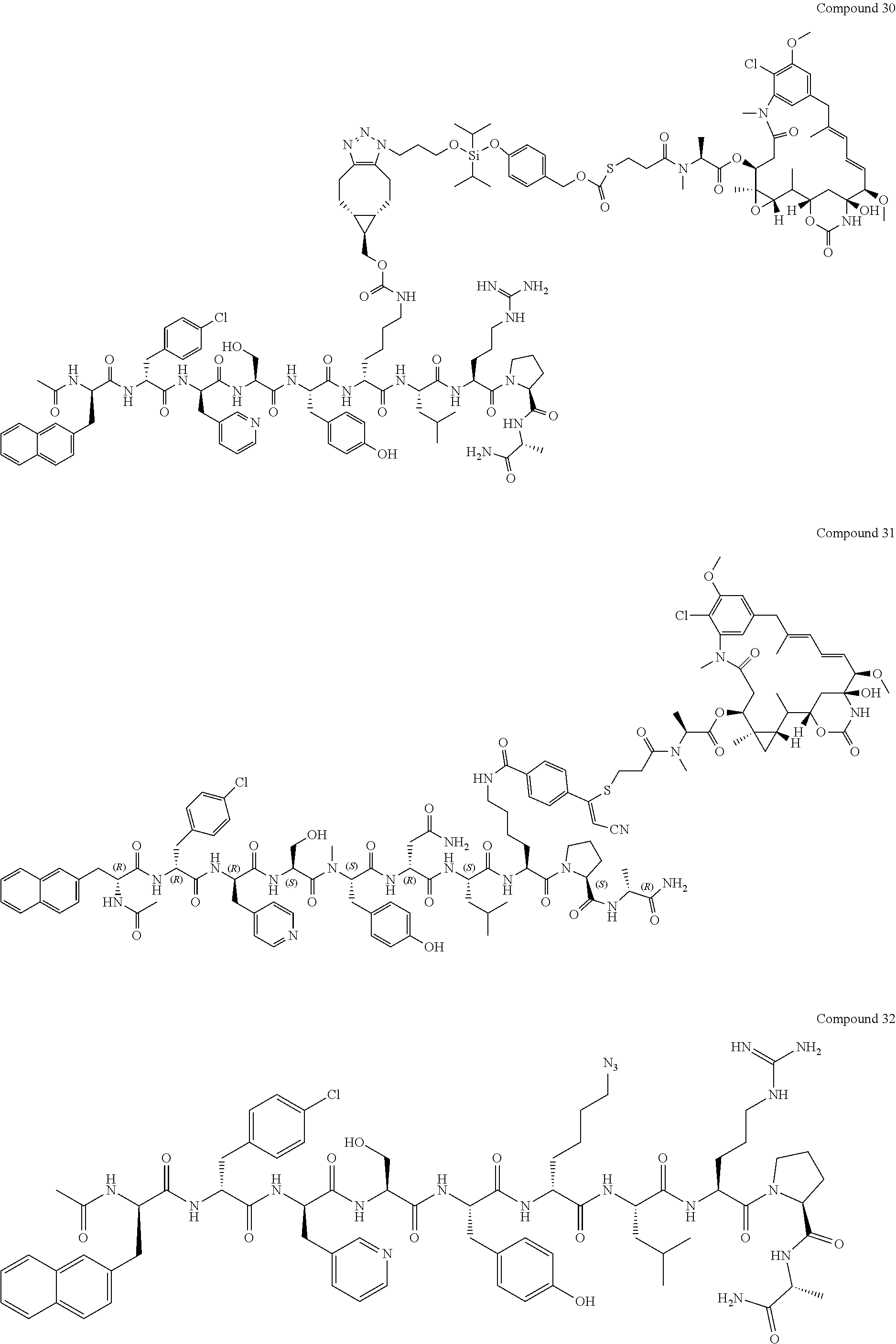

Preferred compounds are Compounds 8, 16, 27, and 28.

The term “alkyl” herein refers to a straight or branched, monovalent or bivalent hydrocarbon group, containing 1-20 (e.g., 1-10 and 1-6) carbon atoms. Examples include methyl, ethyl, n-propyl, i-propyl, n-butyl, i-butyl, and t-butyl. Alkyl includes its halo substituted derivatives, i.e., haloalkyl, which refers to alkyl substituted with one or more halogen (chloro, fluoro, bromo, or iodo) atoms. Examples include trifluoromethyl, bromomethyl, and 4,4,4-trifluorobutyl. The term “alkoxy” refers to an —O-alkyl group. Examples include methoxy, ethoxy, propoxy, and isopropoxy. Alkoxy includes haloalkoxy, referring to alkoxy substituted with one or more halogen atoms. Examples include —O—CH₂Cl and —O—CHClCH₂Cl.

The term “cycloalkyl” refers to a saturated and partially unsaturated, monocyclic, bicyclic, tricyclic, or tetracyclic, monovalent or bivalent hydrocarbon group having 3 to 12 carbons. Examples of cycloalkyl groups include, but are not limited to, cyclopropyl, cyclobutyl, cyclopentyl, cyclopentenyl, cyclohexyl, cyclohexenyl, cycloheptyl, and cyclooctyl.

The term “heterocycloalkyl” refers to a nonaromatic, monovalent or bivalent, 5-8 membered monocyclic, 8-12 membered bicyclic, or 11-14 membered tricyclic ring system having one or more heteroatoms (e.g., O, N, P, and S). Examples of heterocycloalkyl groups include, but are not limited to, piperazinyl, imidazolidinyl, azepanyl, pyrrolidinyl, dihydrothiadiazolyl, dioxanyl, morpholinyl, tetrahydropuranyl, and tetrahydrofuranyl.

The term “aryl” refers to a monovalent or bivalent, 6-carbon monocyclic, 10-carbon bicyclic, 14-carbon tricyclic aromatic ring system wherein each ring may have 1 to 5 substituents. Examples of aryl groups include phenyl, naphthyl, and anthracenyl. The term “arylene” refers to bivalent aryl. The term “aralkyl” refers to alkyl substituted with an aryl group.

The term “heteroaryl” refers to a monovalent or bivalent, aromatic, 5-8 membered monocyclic, 8-12 membered bicyclic, or 11-14 membered tricyclic ring system having one or more heteroatoms (e.g., O, N, P, and S). Examples include triazolyl, oxazolyl, thiadiazolyl, tetrazolyl, pyrazolyl, pyridyl, furyl, imidazolyl, benzimidazolyl, pyrimidinyl, thienyl, quinolinyl, indolyl, thiazolyl, and benzothiazolyl. The term “heteroaryl alkyl” refers to an alkyl group substituted with a heteroaryl group.

The term “carbonyl” refers to —C(O)—.

The term “halo” refers to a fluoro, chloro, bromo, or iodo radical. The term “amino” refers to a radical derived from amine, which is unsubstituted or mono-/di-substituted with alkyl, aryl, cycloalkyl, heterocycloalkyl, or heteroaryl. The term “alkylamino” refers to alkyl-NH—. The term “dialkylamino” refers to alkyl-N(alkyl)-.

Alkyl, cycloalkyl, heterocycloalkyl, aryl, heteroaryl, aralkyl, heteroaralkyl, alkoxy, and aryloxy mentioned herein include both substituted and unsubstituted moieties. Examples of substituents include, but are not limited to, halo, hydroxyl, amino, cyano, nitro, mercapto, alkoxycarbonyl, amido, carboxy, alkanesulfonyl, alkylcarbonyl, carbamido, carbamyl, carboxyl, thioureido, thiocyanato, sulfonamido, alkyl, alkenyl, alkynyl, alkyloxy, aryl, heteroaryl, cycloalkyl, and heterocycloalkyl, in which alkyl, alkenyl, alkynyl, alkyloxy, aryl, heteroaryl cycloalkyl, and heterocycloalkyl may further substituted.

Compounds of Formula (I), (II), or (III) can include an anion. Examples of an anion include Cl⁻, Br⁻, I⁻, SO₄²⁻, PO₄³⁻, ClO₄⁻, CH₃CO₂⁻, and CF₃CO₂⁻.

The term “compound”, when referring to a compound of Formula (I), (II), or (III), also covers its salts, solvates, and prodrugs. A salt can be formed between an anion and a positively charged group (e.g., amino) on a compound; examples of a suitable anion include chloride, bromide, iodide, sulfate, nitrate, phosphate, citrate, methanesulfonate, trifluoroacetate, acetate, malate, tosylate, tartrate, fumurate, glutamate, glucuronate, lactate, glutarate, and maleate. A salt can also be formed between a cation and a negatively charged group; examples of a suitable cation include sodium ion, potassium ion, magnesium ion, calcium ion, and an ammonium cation such as tetramethylammonium ion. Further, a salt can contain quaternary nitrogen atoms. A solvate refers to a complex formed between an active compound and a pharmaceutically acceptable solvent. Examples of a pharmaceutically acceptable solvent include water, ethanol, isopropanol, ethyl acetate, acetic acid, and ethanolamine. A prodrug refers to a compound that, after administration, is metabolized into a pharmaceutically active drug. Examples of a prodrug include esters and other pharmaceutically acceptable derivatives, which, upon administering to a subject, are capable of providing active compounds of this invention.

The details of the invention are set forth in the drawings, the definitions, and the detailed description below. Other features, objects, and advantages of the invention will be apparent from the following actual examples and claims.

### DETAILED DESCRIPTION OF THE INVENTION

As disclosed above, LHRH-drug conjugates are used to treat certain cancer.

Luteinizing hormone releasing hormone (also known as “gonadotropin-releasing hormone” or “GNRH”) or “LHRH” is hormone that is a decapeptide having the following structure: (Pyr)-Glu-His-Trp-Ser-Tyr-Gly-Leu-Arg-Pro-Gly-NH₂.

LHRH is used as a ligand in an LHRH-conjugate of this invention. It attached to a drug moiety though one or more linker groups.

An LHRH analogue is also suitable as a ligand for LHRH-drug conjugates of this invention, Such an analogue includes LHRH (or GNRH) agonist, a LHRH (or GNRH) antagonist or any combination of a LHRH analogue, LHRH agonist or LHRH antagonist that is capable of binding to the LHRH receptor, Preferably, the LHRH analogue, LHRH agonist, LHRH antagonist or any combination thereof is capable of binding to one or more LHRH receptors and are gonadotropin secretory inhibitors or gonadotropin receptor effect blockers.

LHRH agonists that can be used in the present invention, for example, include the peptides described in Treatment with LHRH analogs: Controversies and perspectives. The Parthenon Publishing Group Ltd. (1996), JP-A-3-503165, JP-A-3-101695, JP-A-7-97334 and JP-A-8-259460 and the like. An exemplary peptide useful in the present invention has the following formula:

(Pyr)Glu-R^{1a}-Trp-Ser-R^{2a}—R^{3a}—R^{4a}-Arg-Pro-R^{5a} (IA)

wherein R^{1a} is His, Tyr, Trp or p-NH₂-Phe; R^{2a} is Tyr or Phe; R^{3a} is Gly or D type amino acid residue that may optionally have one or more substituents; R^{4a} is Leu, Ile or Nle; and R^{5a} is Gly-NH—R^{6a} (R^{6a} is a hydrogen atom or an alkyl group optionally having a hydroxyl group), NH—R^{7a} (R^{7a} is a hydrogen atom, an amino group, an alkyl group optionally having a hydroxyl group, or an ureido group (—NH—CO—NH₂)), or a salt thereof.

In the aforementioned formula (IA), when R^{3a} is a D type amino acid residue, said D type amino acid can be an α-D-amino acid having up to 9 carbon atoms (i.e., D-Leu, Ile, Nle, Val, Nval, Abu, Phe, Phg, Ser, Thr, Mot, Ala, Trp, α-Aibu) or the like. Examples of the substitutents that can be used with R^{3a}, include, but are not limited to, tert-butyl, tert-butoxy, tert-butoxycarbonyl, methyl, dimethyl, trimethyl, 2-naphthyl, indolyl-3-yl, 2-methylindolyl, benzyl-imidazo-2-yl and the like. Additionally in formula (I), examples of an alkyl group for R^{6a} or R^{7a}, include, but are not limited to, a C₁₋₄ alkyl group, which is exemplified by methyl, ethyl, propyl, isopropyl, butyl, isobutyl, sec-butyl and tert-butyl.

In addition, a salt of the peptide represented by the formula (IA) (which is also referred to as “peptide (IA)” herein), include, but are not limited to, an acid salt (i.e., carbonate, bicarbonate, acetate, trifluoroacetate, propionate, succinate etc.) and a metal complex compound (i.e., copper complex, zinc complex etc.) are used. Peptide (IA) or a salt thereof can be produced using any method known to those skilled in the art, such as a method described in, for example, U.S. Pat. Nos. 3,853,837, 4,008,209, 3,972,859, GB patent No. 1,423,083, Proceedings of the National Academy of Sciences of the United States of America, vol. 78, pp. 6509-6512 (1981) and the like or a method analogous thereto.

Preferably, peptide (IA) can have a structure shown in US 2012/0129773 A1, especially formulas (a)-(j).

LHRH antagonists that can be used in the present invention can, for example, include those disclosed in U.S. Pat. Nos. 4,086,219, 4,124,577, 4,253,997 and 4,317,815, or a peptide represented by formula II of US 2012/0129773 A1. These peptides include their salts and can be used an optical isomer, or a mixture of the optical isomers. A pharmacologically acceptable salt is preferably used. Examples of such salts, include, but are not limited to, salts of inorganic acids (i.e., hydrochloric acid, sulfuric acid, nitric acid and the like), salts of organic acids (i.e., carbonic acid, bicarbonic acid, succinic acid, acetic acid, propionic acid, trifluoroacetic acid and the like) and the like. Preferably, the salt of the peptide is a salt of an organic acid (i.e., carbonic acid, bicarbonic acid, succinic acid, acetic acid, propionic acid, trifluoroacetic acid and the like). Most preferably, the salt of the peptide is a salt of acetic acid. More specifically, these salts can be mono, di or tri salts.

The peptide or a salt thereof can be produced by any method known to those skilled in the art, such as a method described in JP-A-3-101695 (EP-A 413209), Journal of Medicinal Chemistry, Vol. 35, p. 3942 (1992) and the like, or a method analogous thereto.

Additionally, it is possible to use a linear peptide which is a derivative of LHRH (U.S. Pat. Nos. 5,140,009 and 5,171,835), a cyclic hexapeptide derivative (JP-A-61-191698), a bicyclic peptide derivative (Journal of Medicinal Chemistry, Vol. 36, pp. 3265-3273 (1993)) and the like. Examples of non-peptide compounds having an LHRH antagonistic action, compounds described in JP-A-62-116514, WO 95/28405 (JP-A-8-295693), WO 97/14697 (JP-A-9-169767), WO 97/14682 (JP-A-9-169735), WO 96/24597 (JP-A-9-169768), J. Med. Chem., Vol. 32, pp. 2036-2038 (1989) and the like can be used.

Examples of suitable LHRH antagonists include, but are not limited to, abarelix, ganirelix, cetrorelix, 5-(N-benzyl-N-methyl-aminomethyl)-1-(2,6-difluorobenzyl)-6-[4-(3-methoxyureido)phenyl]-3-phenylthieno-[2,3-d]pyrimidine-2,4(1H,3H)-dione, 5-(N-benzyl-N-methylaminomethyl)-1-(2,6-difluorobenzyl)-6-[4-(3-ethylureido)phenyl]-3-phenylthieno[2,3-d]pyrimidine-2,4(1H,3H)-dione and 5-(N-benzyl-N-methylaminomethyl)-1-(2,6-difluorobenzyl)-6-[4-(3-ethylureido)phenyl]-3-phenylthieno[2,3-d]pyrimidine-2,4(1H,3H)-dione hydrochloride.

Any anticancer drug can be attached to the LHRH ligand through a crosslinker. Examples of an anticancer drug include oxaliplatin platinum salts, vinca alkaloids, eribulin, epothilones (e.g., ixabepilone available as Ixempra® from Bristol-Myers Squibb, New York, New York), arsenic trioxide (Trisenox®, Teva Pharmaceuticals USA, Parsippany, New Jersey), cytarabine (Cytosar-U® and Depocyt®), etoposide, hexamethylmelamine, ifosfamide (Ifex@), methotrexate (Trexall®), procarbazine (Matulane®), vinblastine, platinum compounds (cisplatin, carboplatin, oxaliplatin), vincristine, taxanes (docetaxel, paclitaxel), bortezomib (Velcade®), thalidomide (Thalomid®), and lenalidomide.

The LHRH-drug conjugates are useful in treating different cancers (e.g., colorectal cancer, renal cell carcinoma, lung cancer, pancreatic cancer, endometrial cancer, ovarian cancer, breast cancer, and prostate cancer).

LHRH (or its analogues) is attached covalently to a drug moiety through one or more linker moieties. A crosslinker is a chemical reagent that has two or more reactive groups at its ends. Examples of reactive groups found in a crosslinker include, but are not limited to, amine for conjugation with the carboxylate group of a target molecule; NHS-Ester for the amine group of a target molecule; maleimide for the thiol group of a target molecule; isocyanate for the hydroxyl group of a target molecule; alkyne (—C≡CH) for the azide group (—N3) of a target molecule; hydrazine or aminooxy for the aldehyde or keto group of a target molecule, respectively. When the crosslinker reacts with both LHRH (or its analogue) and a drug molecule, it bonds to LHRH (or its analogue) at one end and the drug moiety at the other end.

The crosslinker moiety of an LHRH-drug conjugate preferably contains cleavable groups such as —NHC(O)O—, —NHC(O)N—, —NHC(O)—, —C(O)O—, —C(O)S—, —S—S—, and an amino acid moiety. Examples are shown in the supra drug-linker moieties.

Preparation of compounds of Formula (I), (II), or (III) can be achieved by reacting a crosslinker, a drug molecule, and LHRH (or an analogue) stepwise or in one pot using conventional methods, e.g., procedures provided in the references cited above. Their antitumor activities are then evaluated using known methods in the art.

Compounds of this invention contain a non-aromatic double bond or one or more asymmetric centers. Each of them occurs as a racemate or a racemic mixture, a single R enantiomer, a single S enantiomer, an individual diastereomer, a diastereomeric mixture, a cis-isomer, or a trans-isomer. Compounds of such isomeric forms are within the scope of this invention. They can be present as a mixture or can be isolated using chiral synthesis or chiral separation technologies.

A compound of Formula (I), (II), or (III) is preferably formulated into a pharmaceutical composition containing a pharmaceutical carrier. The composition is then given to a subject in need thereof to treat a LHRH receptor-associated cancer.

To practice the method of the present invention, a composition having one or more of the above-described conjugates can be administered parenterally, orally, nasally, rectally, topically, or buccally.

The term “parenteral” as used herein encompasses subcutaneous, intracutaneous, intravenous, intraperitoneal, intramuscular, intraarticular, intraarterial, intrasynovial, intrasternal, intrathecal, intralesional, and intracranial injection of a sterile injectable composition. Indeed, the term refers to any suitable infusion technique.

A sterile injectable composition can be a solution or suspension in a non-toxic parenterally acceptable diluent or solvent, such as a solution in 1,3-butanediol. Among the acceptable vehicles and solvents that can be employed are mannitol, water, Ringer's solution, and isotonic sodium chloride solution. In addition, fixed oils are conventionally employed as a solvent or suspending medium (e.g., synthetic mono- or di-glycerides). Fatty acid, such as oleic acid and its glyceride derivatives are useful in the preparation of injectables, as are natural pharmaceutically acceptable oils, such as olive oil and castor oil, especially in their polyoxyethylated versions. These oil solutions or suspensions can also contain a long chain alcohol diluent or dispersant, carboxymethyl cellulose, or similar dispersing agents. Other commonly used surfactants such as Tweens and Spans or other similar emulsifying agents or bioavailability enhancers which are commonly used in the manufacture of pharmaceutically acceptable solid, liquid, or other dosage forms can also be used for the purpose of formulation.

A composition for oral administration can be any orally acceptable dosage form including capsules, tablets, emulsions and aqueous suspensions, dispersions, and solutions. In the case of tablets, commonly used carriers include lactose and corn starch. Lubricating agents, such as magnesium stearate, are also typically added. For oral administration in a capsule form, useful diluents include lactose and dried corn starch. When aqueous suspensions or emulsions are administered orally, the active ingredient can be suspended or dissolved in an oily phase combined with emulsifying or suspending agents. If desired, certain sweetening, flavoring, or coloring agents can be added. Oral solid dosage forms can be prepared by spray dried techniques; hot melt extrusion strategy, micronization, and nano milling technologies.

A nasal aerosol or inhalation composition can be prepared according to techniques well known in the art of pharmaceutical formulation. For example, such a composition can be prepared as a solution in saline, employing benzyl alcohol or other suitable preservatives, absorption promoters to enhance bioavailability, fluorocarbons, and/or other solubilizing or dispersing agents known in the art. A composition having an active compound can also be administered in the form of suppositories for rectal administration.

The carrier in the pharmaceutical composition must be “acceptable” in the sense that it is compatible with the active ingredient of the composition (and preferably, capable of stabilizing the active ingredient) and not deleterious to the subject to be treated. One or more solubilizing agents can be utilized as pharmaceutical excipients for delivery of an active compound. Examples of other carriers include colloidal silicon oxide, magnesium stearate, cellulose, sodium lauryl sulfate, and D&C Yellow #10.

The term “treating” refers to application or administration of the compound to a subject with the purpose to cure, alleviate, relieve, alter, remedy, improve, or affect the disease, the symptom, or the predisposition. “An effective amount” refers to the amount of the compound which is required to confer the desired effect on the subject. Effective amounts vary, as recognized by those skilled in the art, depending on route of administration, excipient usage, and the possibility of co-usage with other therapeutic treatments such as use of other active agents. Dosage levels of a compound of Formula (I), (II), or (III) are of the order of 0.01 mg/kg body weight to 500 mg/kg body weight (e.g., 0.05 mg/kg body weight to 300 mg/kg body weight, 0.1 mg/kg body weight to 200 mg/kg body weight, and 1 mg/kg body weight to 100 mg/kg body weight) per day. The specific dose level for a particular patient will depend upon a number of factors including age, body weight, general health, sex, diet, time of administration, rate of excretion, and the severity of the disorder. To enhance the therapeutic efficiency, the compound can be administered concomitantly with one or more of other orally active antitumor compounds.

Without further elaboration, it is believed that one skilled in the art can, based on the above description, utilize the present invention to its fullest extent. The following specific examples are, therefore, to be construed as merely illustrative, and not limitative of the remainder of the disclosure in any way whatsoever.

All publications, including patent documents, cited herein are incorporated by reference in their entirety.

### EXAMPLES

Compounds 1-32 of this application were prepared following the procedures described below.

Materials and agents used in the preparation are commercially available from various suppliers. They are listed below.

Protecting agents: Fmoc-D-Ala-OH, Fmoc-Pro-OH, Fmoc-hArg(Et)₂-OH, Fmoc-D-hArg(Et)₂-OH, Fmoc-Arg(pbf)-OH, Fmoc-D-Arg(pbf)-OH, Fmoc-Leu OH, Fmoc-D-Lys(Boc)-OH, Fmoc-Tyr(tBu)-OH-Fmoc-Ser(tBu)-OH-Fmoc-(D-3-Pal]-OH, Fmoc-D-Phe (4-CI)-OH, and Fmoc-D-2-NAL-OH.

Coupling reagents: HOBT, DIC, DIEA, ACE, CHOH, DCM, and DMF.

Deprotecting reagents: 20% Pip/DMF.

Resin for peptide synthesis and purification: Rink Resin.

### Methods: General Production Process

### 1. Resin Swelling

Add the resin to the reactor and add appropriate amount of DCM to stir and swell for 15-30 min.

### 2. Deprotection

Drain the DCM, add 20% Pip/DMF and stir for 20-30 min.

### 3. Deprotection Washing

Drain the deprotection reagent and wash it 5-8 eq with DMF.

### 4. Deprotection Detection

Put 15-30 pieces of resin into the test tube, then add 1-2 ml detection reagent, put the test tube into. the water bath above 95° C. for 30-60s, take out the test tube and observe the resin color. The color of the resin turns dark, indicating successful deprotection.

### 5. Coupling of the First Amino Acid

Appropriate amount of DM was added to dissolve 3-5 eq FMOC-D-ALA-OH and HOBI, and then. 3-5 eg DIC was added to stir the reaction for 1-2 h.

### 6. Resin Sealing Side

The coupling solution was drained and DMF was added to the reactor, acetic anhydride and DIEA were stirred for 20-30 min.

### 7. Reaction Washing

Drain the reagent and wash it 3-5 eq with DMF.

### 8. Deprotection

Add 20% Pip/DMF and stir for 20-30 min.

### 9. Deprotection Washing

Drain the deprotection reagent and wash it 5-8 eq with DMF.

### 10. Deprotection Detection

Put 15-30 pieces of resin into the test tube, then add 1-2 ml detection reagent, put the test tube into the water bath above 95° C. for 30-60s, take out the test tube and observe the resin color. The color of the resin turns dark, indicating successful deprotection.

### 11. Coupling

Appropriate amount of DM was added to dissolve 3-5 eq Fmoc-Pro-OH and HOBT, and then 3-5 equivalence of DIC was added to stir the rection for 1-2 hr.

### 12. Coupling Detection

Put 15-30 pieces of resin into the test tube, then add 1-2 ml detection reagent, put the test tube into the water bath above 95° C. for 30-60s, take out the test tube and observe the resin color. No obvious change in resin color indicated that the coupling was successful.

Repeat steps 7 to 12 to condense the amino acids in the sequence from C side to N side.

After the coupling of the last amino acid, the deprotection was successful, the resin was washed with appropriate amount of methanol, and was drained for 3-5 h.

### Cleavage Process

Reagent formula: TFA/EDT/water=90/5/5, Stir cracking at room temperature for 2-3 hours, filter out the resin, and add ether to the lysate to precipitate, centrifuge to remove the upper liquid reagent, and then add ether to stir and disperse evenly. Centrifuge 2-3 times and transfer the solid and coarse polypeptide into a clean tray and put it into a vacuum drying oven at 35° Cf or 3-5 h. A small portion of the sample was analyzed by HPLC and MS for purification.

### Purification Process

A quantitative crude peptide was taken and dissolved by ultrasonically adding a quantitative mixture of pure water and acetonitrile. After the sample was clarified and transparent, it was filtered by a vacuum filter and purified by a DAC system.

### Lyophilization Process

After purification, the qualified fraction is loaded into the lyophilized dish, covered and put into the freeze dryer for lyophilization. After lyophilization, the lyophilization plate was taken out, and the polypeptide samples were weighed and divided, and stored at −20° C.

### Synthesis of Peptido-Alkyne Compounds:

Compounds 2A, 6A, 10A, 12A, 13A and 15A were prepared following the procedure described below.

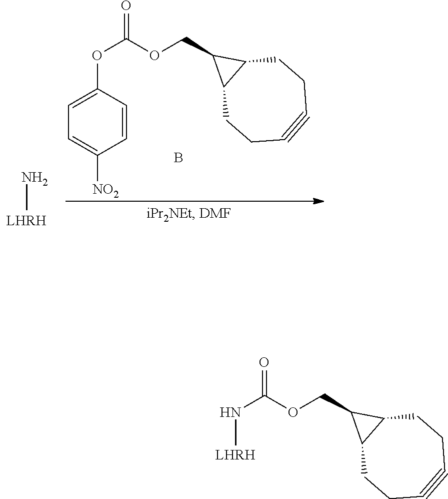

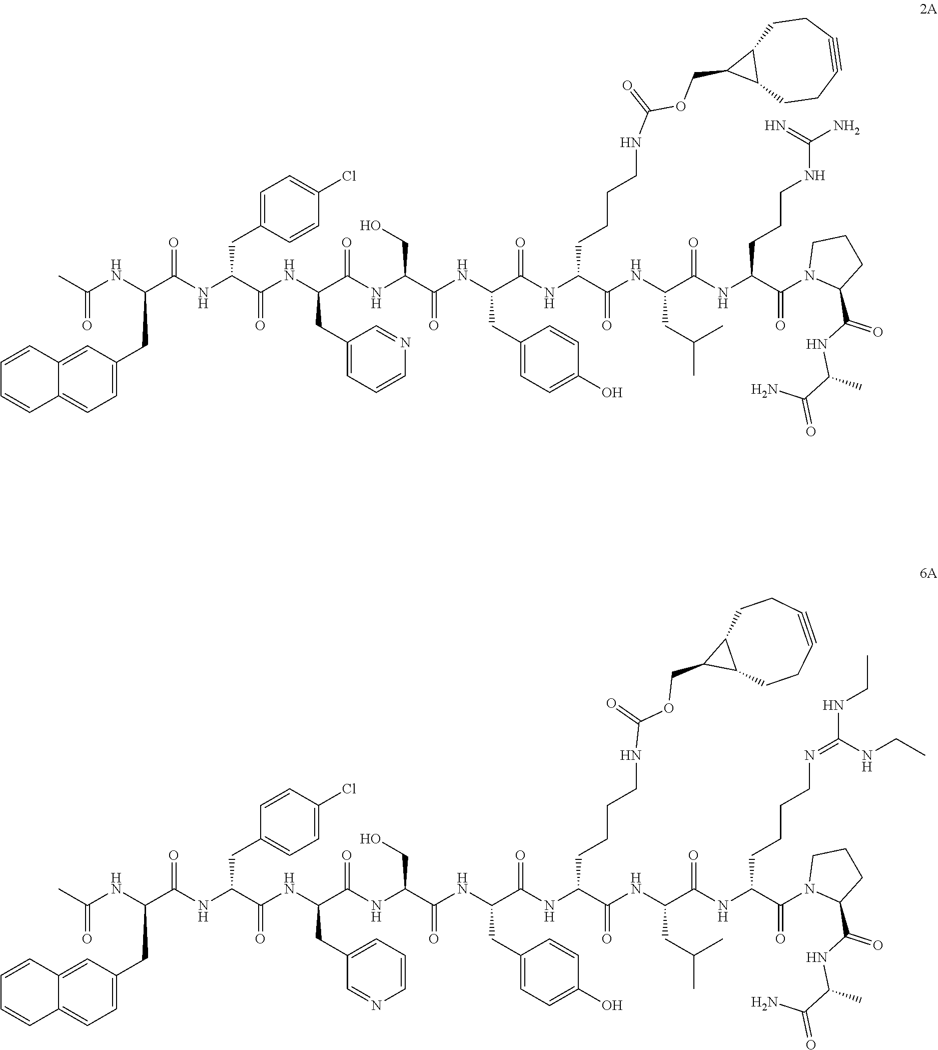

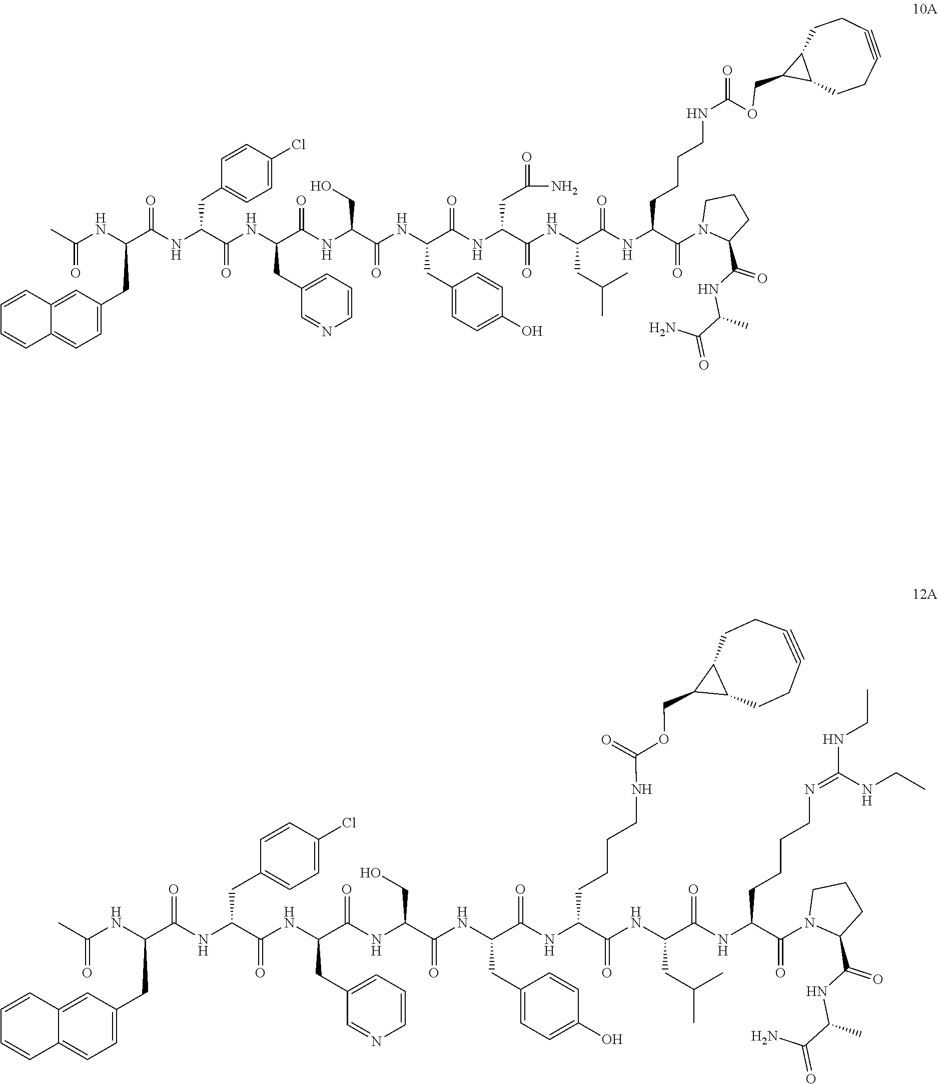

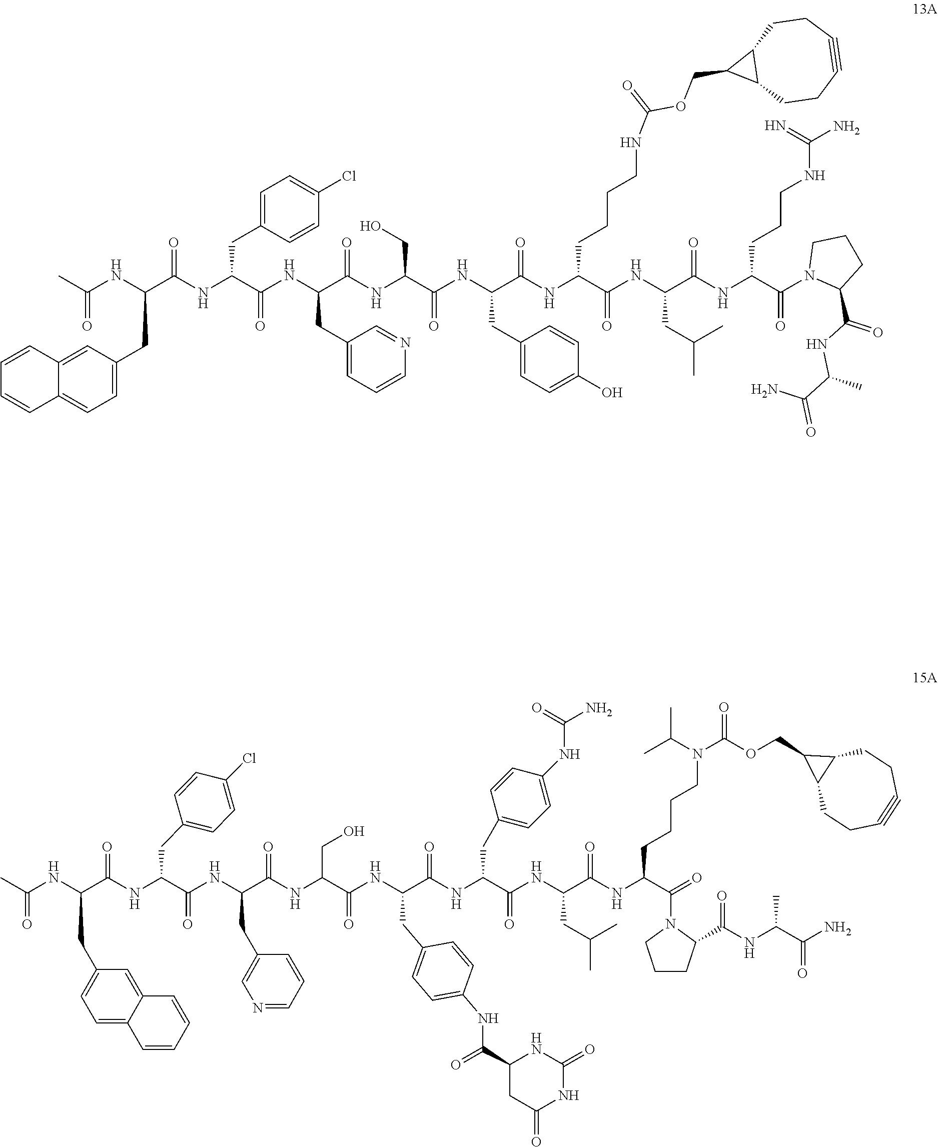

General procedure: To a solution of ((1R,8S,9r)-bicyclo[6.1.0]non-4-yn-9-yl)methyl (4-nitrophenyl) carbonate B (1.05 eq) in DMF (1 mL) was added LHRH analogs or Degarelix (1 eq) and DIPEA (3 eq) and the reaction mixture was stirred at rt for 30 min. Completion of the reaction was checked by HPLC. The reaction mixture was concentrated under reduced pressure. The residual oil was treated with ethyl acetate to precipitate a solid, which was purified by washing with ether, EA, CH₂Cl₂ to afford a peptido alkyne compound as a white solid.

### Synthesis of Peptido-Azide Compounds:

Compound 32 was prepared as depicted in the scheme below, following a procedure described in Gironda-Martínez et al., J. Org. Lett. 2019, 21, 9555.

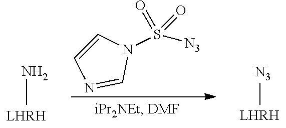

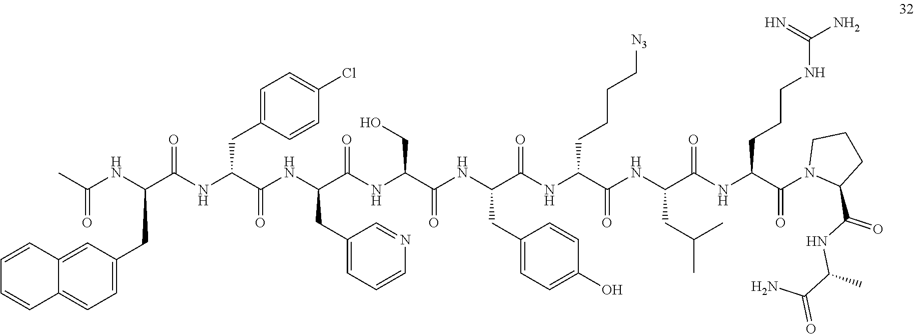 [SMILES: CC(=O)N[C@H](Cc1ccc2ccccc2c1)C(=O)N[C@H](Cc1ccc(Cl)cc1)C(=O)N[C@H](Cc1cccnc1)C(=O)N[C@@H](CO)C(=O)N[C@@H](Cc1ccc(O)cc1)C(=O)N[C@H](CCCCN=[N+]=[N-])C(=O)N[C@@H](CC(C)C)C(=O)N[C@@H](CCCNC(=N)N)C(=O)N1CCC[C@H]1C(=O)N[C@H](C)C(N)=O]

More specifically, LHRH or an analog (1 eq) was dissolved in DMF (1 mL) at 0° C. Imidazole-1-sulfonyl azide hydrochloride (10 eq) and DIPEA (3 eq) were added. The reaction mixture and stirred for 0.5 hours. The solvent was removed in vacuo to obtain an oil, which was treated with ethyl acetate. A resultant solid was collected and purified by washing with ether, EA, CH₂Cl₂ to afford the peptido azide compound (68.4 mg, 67.5%) as a white solid. LC-MS: [M+H⁺]⁺=1429.

### Synthesis of Linker-Drugs A to L

### Linker-Drug A

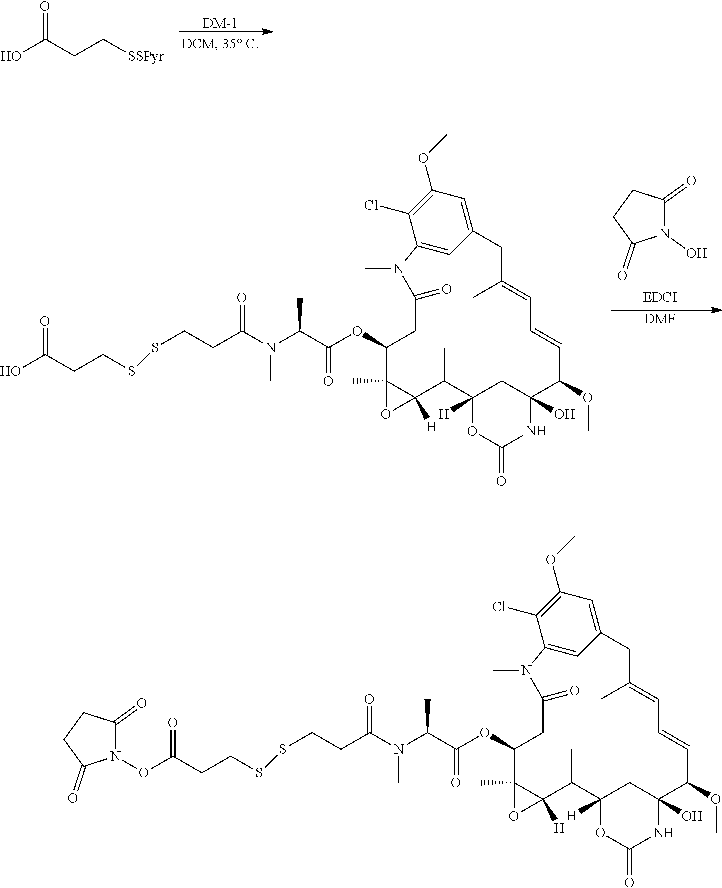

Linker-drug A was prepared following a procedure described in US Application Publication No. 2022/0401592 A1.

### Linker-Drug B

Linker-drug B was prepared following a procedure described International Application Publication No. WO2021262628 A1.

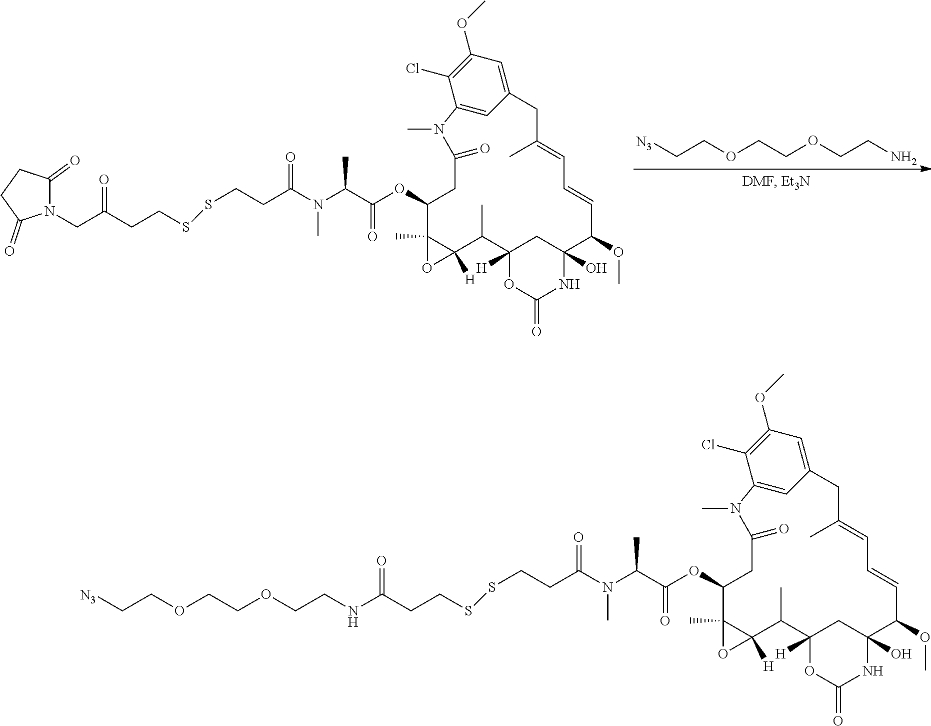 [SMILES: COc1cc2cc(c1Cl)N(C)C(=O)C[C@H](OC(=O)[C@H](C)N(C)C(=O)CCSSCCC(=O)CN1C(=O)CCC1=O)[C@]1(C)O[C@H]1C(C)[C@@H]1C[C@@](O)(NC(=O)O1)[C@H](OC)/C=C/C=C(\C)C2 ; [N-]=[N+]=NCCOCCOCCN ; COc1cc2cc(c1Cl)N(C)C(=O)C[C@H](OC(=O)[C@H](C)N(C)C(=O)CCSSCCC(=O)NCCOCCOCCN=[N+]=[N-])[C@]1(C)O[C@H]1C(C)[C@@H]1C[C@@](O)(NC(=O)O1)[C@H](OC)/C=C/C=C(\C)C2]

Linker-drug A (40 mg, 1 eq) was dissolved in DMF (1.0 mL), 2-(2-(2-azidoethoxy) ethoxy) ethan-1-amine (8.15 mg, 1.1 eq) and Et₃N (12.9 mg, 3 eq). The resultant reaction mixture was stirred for 1 hour at room temperature. The solvent was removed in vacuo. The crude product was extracted with CH₂Cl₂ and 1M HCl_{(aq)}. The aqueous layer was neutralized. The product was then extracted with CH₂Cl₂. The organic layers were combined, dried with Na₂SO₄, and filtered. The solvent was evaporated to give Linker-drug B (yield=92.4%).

### Linker-Drug C

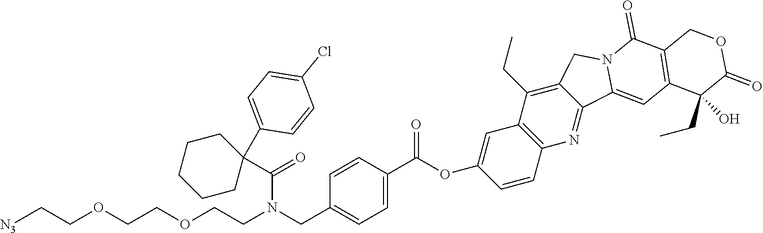 [SMILES: CCc1c2c(nc3ccc(OC(=O)c4ccc(CN(CCOCCOCCN=[N+]=[N-])C(=O)C5(c6ccc(Cl)cc6)CCCCC5)cc4)cc13)-c1cc3c(c(=O)n1C2)COC(=O)[C@@]3(O)CC]

Linker-drug C was prepared following a procedure described in Tsai et al., Front Chem. 2022, 9, 822587.

### Linker-Drug D

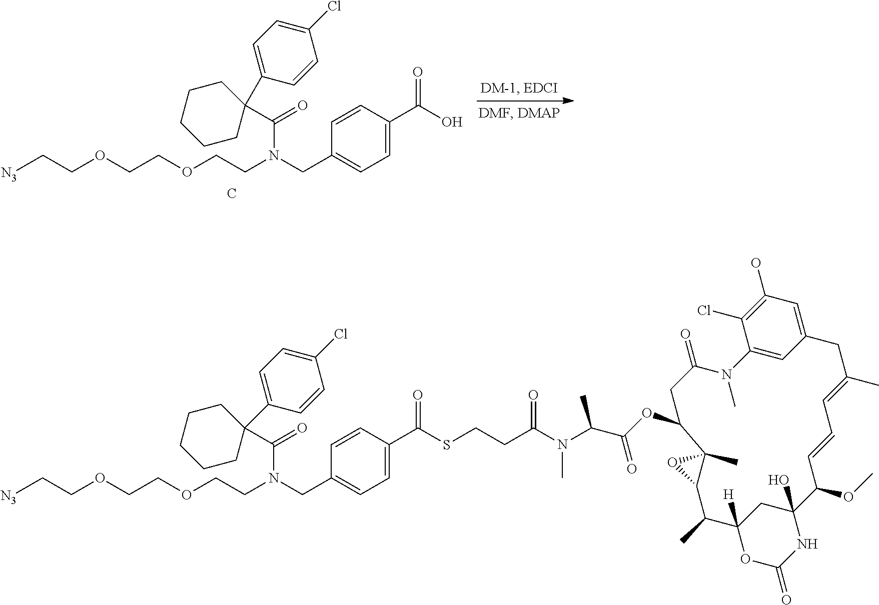

Linker-drug D was prepared following a procedure described in Shih et al., WO2021262628 A1.

More specifically, to a stirred solution of acid C (150 mg, 0.284 mmol, 1 eq) in 5 ml of DMF at room temperature, DM1 (200 mg, 0.271 mmol, 0.95 eq) and EDCI (81.5 mg, 0.425 mmol, 1.5 eq) and DMAP (6.92 mg, 0.06 mmol, 0.2 eq) were slowly added. The resultant reaction mixture was stirred at room temperature for 1 hour, then extracted with CH₂Cl₂ (20 mL). Subsequently, the CH₂Cl₂ solution was washed with a saturated aqueous solution of NaHCO₃ (10 mL) and water (3×10 mL), dried over MgSO₄, and concentrated under reduced pressure. The residue thus obtained was purified by column chromatography (silica gel; EA:Hexane=9:1 to 100% EA) to yield Linker-drug D (280.5 mg, 83%).

### Linker-Drug E

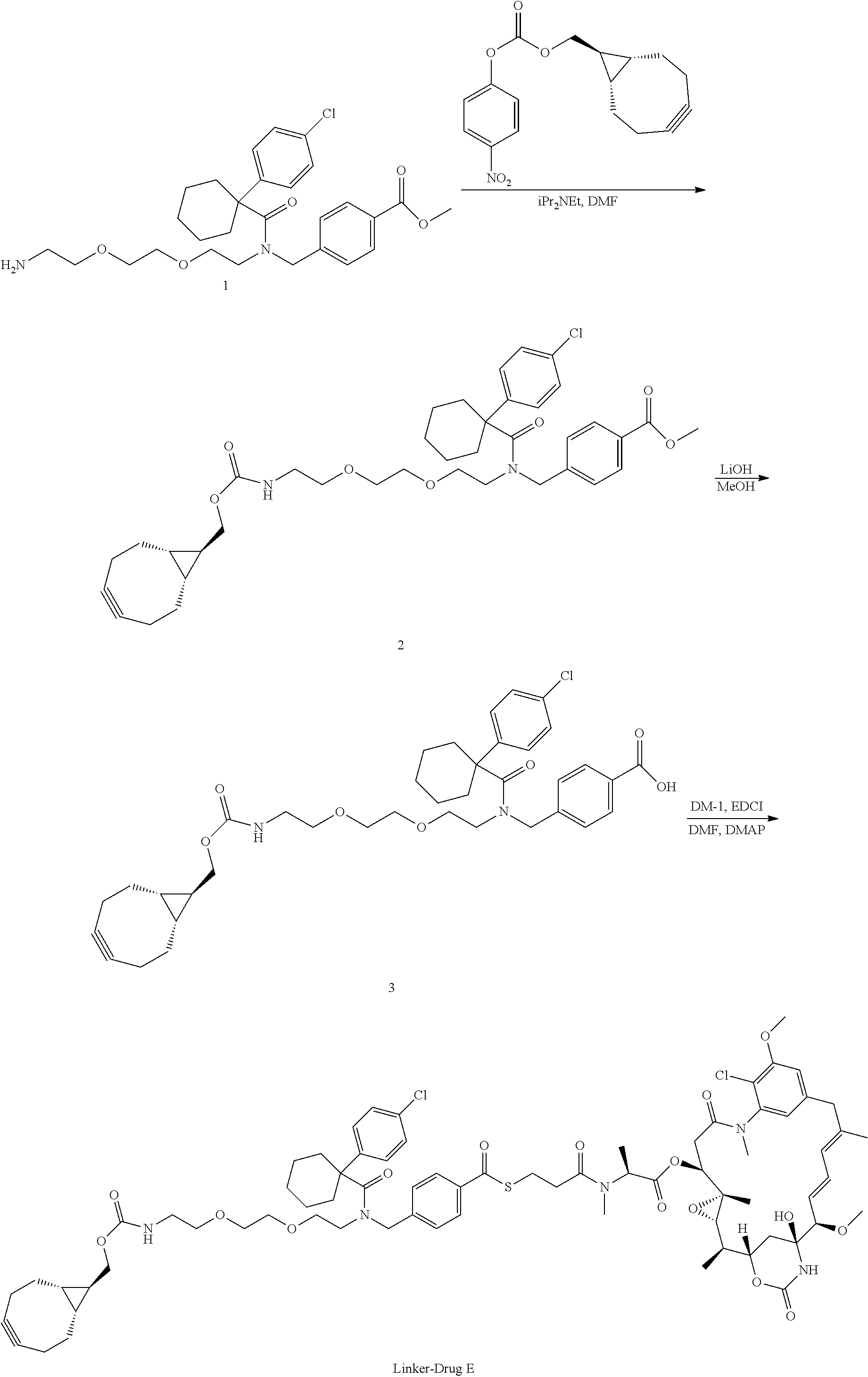

To a solution of ((1R,8S,9r)-bicyclo[6.1.0]non-4-yn-9-yl)methyl (4-nitrophenyl) carbonate B (93.9 mg, 1 eq) in DMF (1 mL) was added compound 1 (154 mg, 1 eq) (Tsou et al., WO2021262628 A1) and DIPEA (3 eq). The reaction mixture was stirred at rt for 30 min. The reaction was complete by checking HPLC, and then concentrated under reduced pressure. The residue was purified by column chromatography (silica gel; EA:Hexane=1:6 to 1:1) to yield compound 2 (Yield=80.6%). Then the product was dissolved in MeOH, and added 6N LiOH (aq) at room temperature. The solvent was removed and concentrated under reduced pressure. And the residue was redissolved in CH₂Cl₂. The insoluble residue filtered off. The filtrate was washed with water, dried over MgSO₄₍ₛ₎ and the solvent removed under vacuum. The product acid 3 was obtained as white foam product and check by LC-MS ([M+Na⁺]⁺=702).

To a stirred solution of acid 3 (120 mg, 0.177 mmol, 1 eq) in 5 ml of DMF at room temperature, DM1 (195.65 mg, 0.265 mmol, 1.5 eq) and EDCI (67.7 mg, 0.353 mmol, 2.0 eq) and DMAP (23.7 mg, 0.194 mmol, 1.1 eq) were slowly added and stirred at room temperature for 1 hours. The resultant residue was extracted with CH₂Cl₂ (10.0 mL). The CH₂Cl₂ solution was then washed with a saturated aqueous solution of NaHCO₃ (5.0 mL) and water (3×5.0 mL), dried over MgSO₄, and concentrated under reduced pressure. The resultant residue was purified by column chromatography (silica gel; EA:Hexane=9:1 to 100% EA) to yield Linker-drug E (113.6 mg, 45.8%) and check by LC-MS ([M+H⁺]⁺=1400).

### Linker-Drug F

Linker-drug F was prepared following a known procedure described in Wagner et al., Bioconjugate Chem. 2015, 26, 197-200.

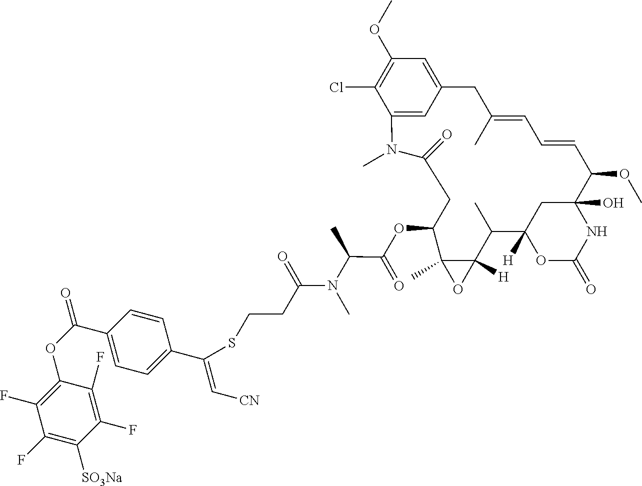

More specifically, CBTF (22.85 mg, 1 eq) and DM1 (40 mg, 1 eq) were dissolved in DMSO (1 mL). The reaction mixture was stirred for 1 hour at room temperature. The crude product was extracted with CH₂Cl₂ and 1N NH₄Cl_{(aq)}. The organic layers were combined, dried with Na₂SO₄, and filtered. Evaporation of the solvent gave Linker-drug F.

LC-MS: [M+Na⁺]⁺=1182.

### Linker-Drug G

Linker-drug G was prepared following the procedure described in WO2021262628 A1.

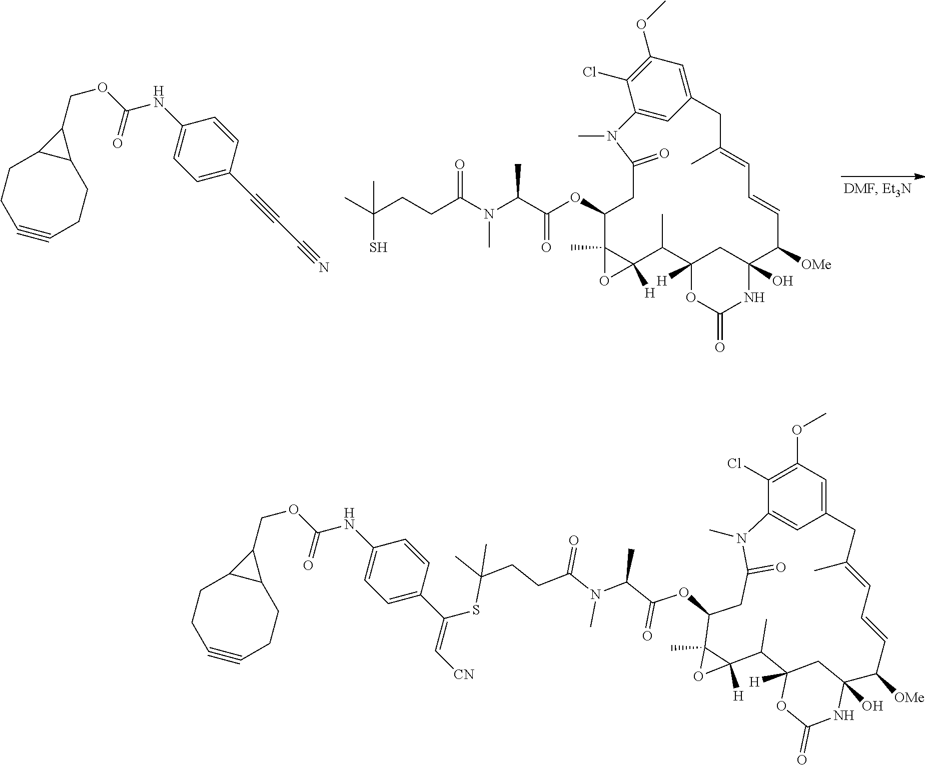

More specifically, to a stirred solution of alkyne (17.1 mg, 0.054 mmol, 1 eq) in 1 mL of DCM/Et₃N (1/1) at room temperature was slowly added DM4 (40 mg, 0.051 mmol, 1 eq). The reaction mixture was stirred at room temperature for 1 hour and then diluted with CH₂Cl₂ (10 mL). The CH₂Cl₂ solution was washed with a saturated aqueous solution of NaHCO₃ (5 mL) and water (3×5 mL), dried over MgSO₄, and concentrated under reduced pressure. The resultant residue was purified by column chromatography (silica gel; EA:Hexane=9:1 to 100% EA) to yield Linker-drug G (22.1 mg, 39.5%).

LC-MS: [M+2H⁺]²⁺=550.

### Linker-Drug H

Linker-drug H was prepared following the procedure described in Lee et al., Journal of Medicinal Chemistry 2008, 51, 6442-49.

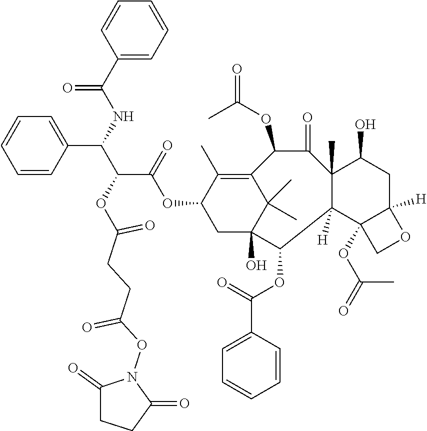 [SMILES: CC(=O)O[C@H]1C(=O)[C@@]2(C)[C@H]([C@H](OC(=O)c3ccccc3)[C@]3(O)C[C@H](OC(=O)[C@H](OC(=O)CCC(=O)ON4C(=O)CCC4=O)[C@@H](NC(=O)c4ccccc4)c4ccccc4)C(C)=C1C3(C)C)[C@]1(OC(C)=O)CO[C@@H]1C[C@@H]2O]

### Linker-Drug I

Linker-drug I was prepared following the procedure described in Cheng et al., WO2015179299 A1.

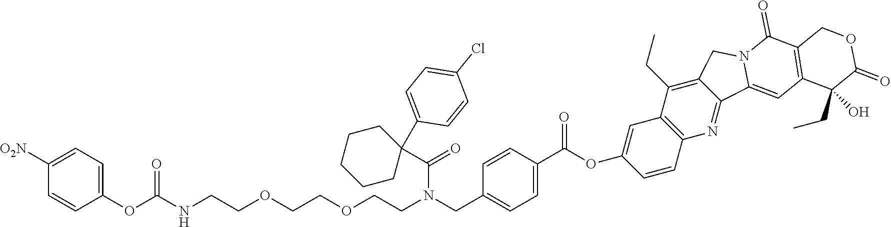 [SMILES: CCc1c2c(nc3ccc(OC(=O)c4ccc(CN(CCOCCOCCNC(=O)Oc5ccc([N+](=O)[O-])cc5)C(=O)C5(c6ccc(Cl)cc6)CCCCC5)cc4)cc13)-c1cc3c(c(=O)n1C2)COC(=O)[C@@]3(O)CC]

### Linker-Drug J

Linker-drug J was prepared following a procedure described in Wang et al., Cancers (2019), 11, 957-974.

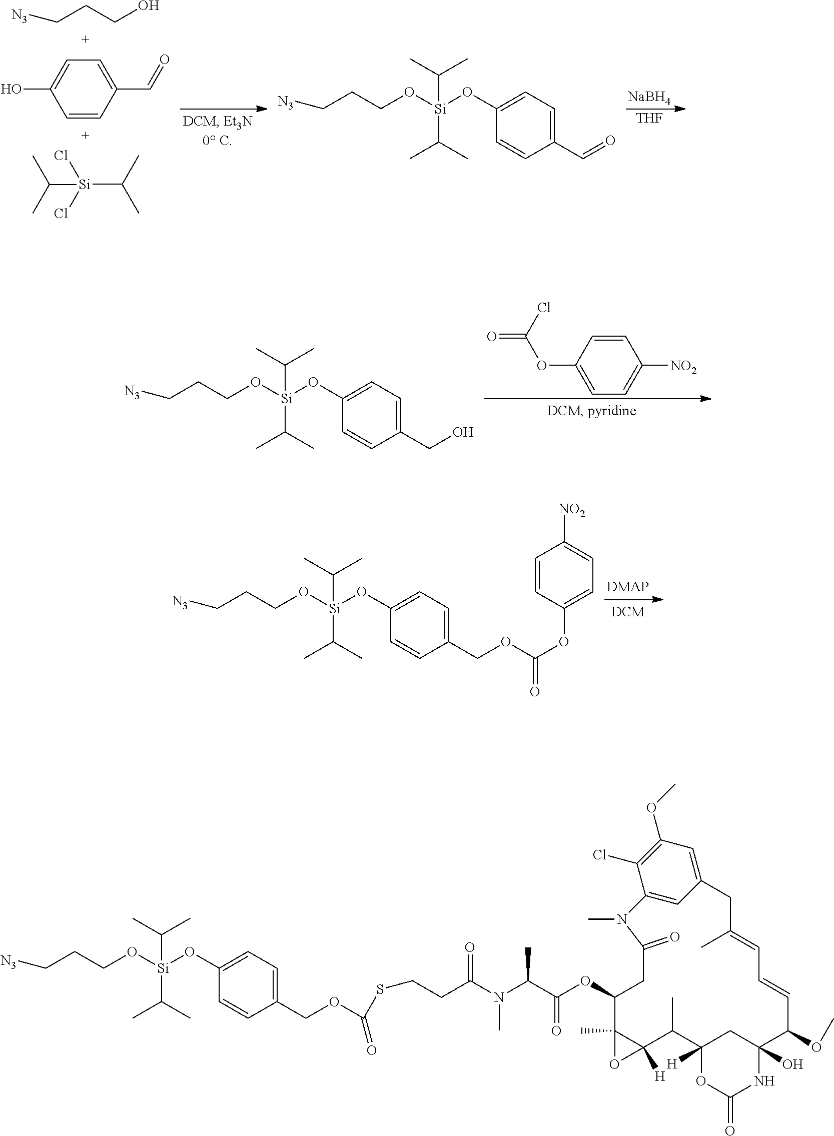

3-Azidopropan-1-ol (1 g, 9.89 mmol, 1 eq) was dissolved in DCM (30 mL) and triethylamine (2 g, 20 mmol, 2 eq) and cooled to 0° C. A solution of dichlorodiisopropylsilane (1.83 g, 10 mmol, 1 eq) in DCM (20 mL) was slowly added dropwise. After 30 min, 4-hydroxybenzaldehyde (1.2 g, 10 mmol, 1 eq) in DCM (10 mL) was slowly added. The reaction mixture was allowed to stir for 1 h. The solvent was concentrated to give a crude product, which was purified by column chromatography to obtain an intermediate aldehyde (2.32 g, 70.1%).

The aldehyde (1.01 g, 3.03 mmol, 1 eq) was dissolved in dry THF (10 mL) and cooled to −5° C. Sodium borohydride (114.6 mg, 3.03 mmol, 1 eq) was added. The mixture was stirred at that temperature for 2 h and then extract with DCM/NH₄Cl. The solvent was removed under reduced pressure to give a crude alcohol, which was purified by column chromatography to obtain the alcohol (0.75 g, 73%) as a colorless oil.

The alcohol (548.3 mg, 1.62 mmol, 1 eq) was dissolved in dry DCM (15 mL), 4-nitrophenyl carbonochloridate (988.5 mg, 3.2 mmol, 2 eq) and DIPEA (315 mg, 2.44 mmol, 1.5 eq), and stirred at room temperature for 2 h. The reaction mixture was extract with DCM/NH₄Cl. The organic layer was separated and concentrated under reduced pressure to a crude product, which was further purified by column chromatography to obtain a PNP-carbonate product (702.7 g, 86.3%) as a white solid.

To a stirred solution of the PNP-carbonate compound (250 mg, 0.497 mmol, 1 eq) in 16 ml of DCM at room temperature was added DM1 (367.2 mg, 0.497 mmol, 1 eq) and DMAP (60.8 mg, 0.4897 mmol, 1 eq). The resultant mixture was stirred at room temperature for 1 hour and then diluted with CH₂Cl₂ (10 mL). The CH₂Cl₂ solution was washed with a saturated aqueous solution of NH₄Cl (5 mL) and water (3×5 mL), dried over MgSO₄, and concentrated under reduced pressure. The resultant residue was purified by column chromatography (silica gel; EA:Hexane=1:1 to 100% EA) to yield Linker-drug J (365.8 mg, 66.8%).

LC-MS: [M+Na⁺]⁺=1124.

### Linker-Drug K

Linker-drug K was prepared following the procedure described in Tang et al., CN110152013 A.

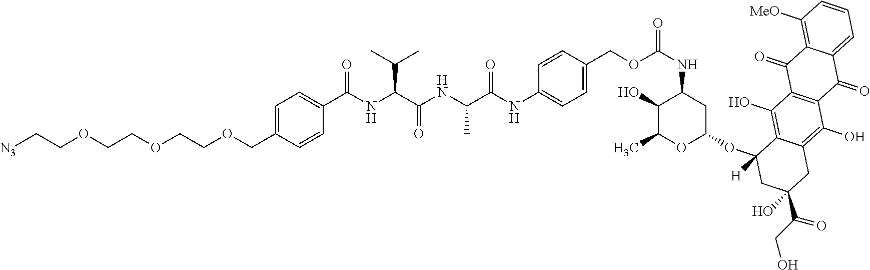 [SMILES: COc1cccc2c1C(=O)c1c(O)c3c(c(O)c1C2=O)C[C@@](O)(C(=O)CO)C[C@@H]3O[C@H]1C[C@H](NC(=O)OCc2ccc(NC(=O)[C@H](C)NC(=O)[C@@H](NC(=O)c3ccc(COCCOCCOCCN=[N+]=[N-])cc3)C(C)C)cc2)[C@H](O)[C@H](C)O1]

### Linker-Drug L

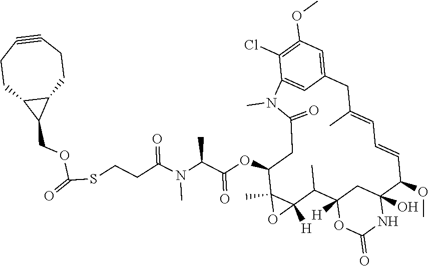 [SMILES: COc1cc2cc(c1Cl)N(C)C(=O)C[C@H](OC(=O)[C@H](C)N(C)C(=O)CCSC(=O)OC[C@H]1[C@@H]3CCC#CCC[C@@H]31)[C@]1(C)O[C@H]1C(C)[C@@H]1C[C@@](O)(NC(=O)O1)[C@H](OC)/C=C/C=C(\C)C2]

A solution of compound B (50 mg, 0.159 mmol, 1 eq), DM-1 (117 mg, 0.16 mmol), DIPEA (48 mg, 0.48 mmol, 3 eq) and DMAP (0.97 mg, 0.008 mmol) in DMF (1 ml) was stirred at RT for 16 hours, then diluted with DCM, and washed several times with a diluted aqueous NaHCO₃. The combined organic layers were dried over Na₂SO₄. The solvent was removed. The resultant residue was purified by column chromatography (silica gel; EA:Hexane=9:1 to 100% EA) and by preparative LC/MS affording Linker-drug L (106.3 mg, 0.083 mmol, 73.1%).

LC-MS: [M+Na⁺]⁺=936.

### Synthesis of Peptide Drug-Conjugates 7-31

Drug conjugates of this invention were prepared by reacting a linker drug (A to L) with a peptide alkyne compound (2A, 6A, 10A, 12A, 13A, and 15A) or peptide azide compound 32.

To a stirred solution of a peptido alkyne compound (1 eq) in DMF at room temperature was added a Linker-drug (1.05 eq). The resultant reaction mixture was stirred at room temperature for 30 min. Completion of the reaction was monitored by HPLC. The solvent was removed under reduced pressure to obtain an oil. Trituration with EA precipitated a crude solid, which was filtered, and washed with EA, Ether, and DCM three times to afford a pure Peptide Drug-Conjugate.

Following the above general procedure, conjugates of this invention was prepared. See Table 1 below including their mass, purity as determined by HPLC, and binding affinity to the LHRH receptor.

Compound 14 was prepared from 6 (18 mg, 0.012 mmol, 1 eq) and Linker-Drug A (11.48 mg, 0.012 mmol, 1 eq) by a general procedure as described for Peptide Drug-Conjugates. Final purification by washing residue with solvents afforded Compound 14 (6.81 mg, 24.7%) as a white solid.

Compound 15 was prepared from 15A (139 mg, 0.0768 mmol, 1 eq) and Linker-Drug C (104 mg, 0.0108 mmol, 1.5 eq) by a general procedure as described for Peptide Drug-Conjugates. Final purification by washing residue with solvents afforded 15 (118 mg, 57%) as a white solid.

Compound 7 was prepared from Compound 4 (49 mg, 0.032 mmol, 1 eq) and Linker-Drug H (33.54 mg, 0.032 mmol, 1 eq) by a general procedure as described for Peptide Drug-Conjugates. Final purification by washing residue with solvents afforded Compound 7 (45.6 mg, 57.6%) as a white solid.

Compound 8 was prepared from Compound 2 (25.2 mg, 0.018 mmol, 1 eq) and Linker-Drug A (16.88 mg, 0.018 mmol, 1 eq) by a general procedure as described for Peptide Drug-Conjugates. Final purification by washing residue with solvents afforded 8 (36.4 mg, 90.8%) as a white solid.

Compound 9 was prepared from Compound 15A (320 mg, 0.177 mmol, 1 eq) and Linker-Drug D (277 mg, 222 mmol, 1.3 eq) by a general procedure as described for Peptide Drug-Conjugates. Final purification by washing residue with solvents afforded 9 (228 mg, 42%) as a white solid.

Compound 10 was prepared from 10A (226.0 mg, 0.146 mmol, 1 eq) and Linker-Drug C (92 mg, 0.102 mmol, 0.7 eq) by a general procedure as described for Peptide Drug-Conjugates. Final purification by washing residue with solvents afforded 10 (164 mg, 46%) as a white solid.

Compound 11 was prepared from 6A (30 mg, 0.018 mmol, 1 eq) and Linker-Drug D (23.86 mg, 0.019 mmol, 1.05 eq) by a general procedure as described for Peptide Drug-Conjugates. Final purification by washing residue with solvents afforded 11 (22.3 mg, 42.8%) as a white solid.

Compound 16 was prepared from 2A (38 mg, 0.024 mmol, 1 eq) and Linker-Drug D (30.06 mg, 0.024 mmol, 1 eq) by a general procedure as described for Peptide Drug-Conjugates. Final purification by washing residue with solvents afforded 16 (27.4 mg, 40.4%) as a white solid.

Compound 18 was prepared from degarelix (133 mg, 0.078 mmol, 1 eq) and Linker-Drug I (90 mg, 0.086 mmol, 1.1 eq) by a general procedure as described for Peptide Drug-Conjugates. Final purification by washing residue with solvents afforded 18 (128 mg, 64%) as a white solid.

Compound 25 was prepared from 2A (30 mg, 0.019 mmol, 1 eq) and Linker-Drug B (18.98 mg, 0.019 mmol, 1 eq) by a general procedure as described for Peptide Drug-Conjugates. Final purification by washing residue with solvents afforded 25 (21.2 mg, 43.3%) as a white solid.

Compound 26 was prepared from 6A (30 mg, 0.018 mmol, 1 eq) and Linker-Drug B (18.18 mg, 0.018 mmol, 1 eq) by a general procedure as described for Peptide Drug-Conjugates. Final purification by washing residue with solvents afforded 26 (24.5 mg, 51.4%) as a white solid.

Compound 29 was prepared from 6A (60 mg, 0.036 mmol, 1 eq) and Linker-Drug J (40.1 mg, 0.036 mmol, 1 eq) by a general procedure as described for Peptide Drug-Conjugates. Final purification by washing residue with solvents afforded 29 (71.3 mg, 72%) as a white solid.

Compound 30 was prepared from 2A (60 mg, 0.038 mmol, 1 eq) and Linker-Drug J (41.8 mg, 0.038 mmol, 1 eq) by a general procedure as described for Peptide Drug-Conjugates. Final purification by washing residue with solvents afforded 30 (61.7 mg, 60.6%) as a white solid.

Compound 17 was prepared from 2A (33 mg, 0.021 mmol, 1 eq) and Linker-Drug K (24.1 mg, 0.021 mmol, 1 eq) by a general procedure as described for Peptide Drug-Conjugates. Final purification by washing residue with solvents afforded 17 (24.4 mg, 42.5%) as a white solid.

Compound 19 was prepared from 15A (60 mg, 0.033 mmol, 1 eq) and Linker-Drug J (36.5 mg, 0.033 mmol, 1 eq) by a general procedure as described for Peptide Drug-Conjugates. Final purification by washing residue with solvents afforded 19 (68.2 mg, 71%) as a white solid.

Compound 20 was prepared from 32 (10 mg, 0.007 mmol, 1 eq) and Linker-Drug L (6.4 mg, 0.007 mmol, 1 eq) by a general procedure as described for Peptide Drug-Conjugates. Final purification by washing residue with solvents afforded 20 (11.6 mg, 70.7%) as a white solid.

Compound 21 was prepared from 32 (28 mg, 0.02 mmol, 1 eq) and Linker-Drug E (27.44 mg, 0.02 mmol, 1 eq) by a general procedure as described for Peptide Drug-Conjugates. Final purification by washing residue with solvents afforded 21 (32.2 mg, 57%) as a white solid.

Compound 22 was prepared from 32 (35 mg, 0.025 mmol, 1 eq) and Linker-Drug G (26.9 mg, 0.025 mmol, 1 eq) by a general procedure as described for Peptide Drug-Conjugates. Final purification by washing residue with solvents afforded 22 (26.1 mg, 41.3%) as a white solid.

Compound 23 was prepared from 12 (60 mg, 0.041 mmol, 1 eq) and Linker-Drug A (38.28 mg, 0.041 mmol, 1 eq) by general procedure as described for Peptide Drug-Conjugates. Final purification by washing residue with solvents afforded 23 (31.7 mg, 33.6%) as a white solid.

Compound 24 was prepared from 13 (30 mg, 0.021 mmol, 1 eq) and Linker-Drug A (20.1 mg, 0.021 mmol, 1 eq) by a general procedure as described for Peptide Drug-Conjugates. Final purification by washing residue with solvents afforded 24 (27.5 mg, 58.8%) as a white solid.

Compound 27 was prepared from 13A (40 mg, 0.025 mmol, 1 eq) and Linker-Drug D (34.81 mg, 0.028 mmol, 1.1 eq) by a general procedure as described for Peptide Drug-Conjugates. Final purification by washing residue with solvents afforded 27 (35.1 mg, 49.1%) as a white solid.

Compound 28 was prepared from 12A (30 mg, 0.018 mmol, 1 eq) and Linker-Drug D (23.89 mg, 0.019 mmol, 1.05 eq) by a general procedure as described for Peptide Drug-Conjugates. Final purification by washing residue with solvents afforded 28 (36.2 mg, 69.4%) as a white solid.

Compound 31 was prepared from Abarelix (20 mg, 0.015 mmol, 1 eq) and Linker-Drug F (16.8 mg, 0.015 mmol, 1 eq) by a general procedure as described for Peptide Drug-Conjugates. Final purification by washing residue with solvents afforded 31 (18.3 mg, 53.9%) as a white solid.

Compound 2A was prepared from 2 (100 mg, 0.071 mmol, 1 eq) and compound B (22.5 mg, 0.019 mmol, 1.05 eq) by a general procedure as described for peptido alkyne adduct. Final purification by washing residue with solvents afforded 2A (86.5 mg, 77.3%) as a white solid. LC-MS: [M+2H⁺]²⁺=790.

Compound 10A was prepared from Abarelix (200 mg, 0.146 mmol, 1 eq) and compound B (69 mg, 0.218 mmol, 1.5 eq) by a general procedure as described for peptido alkyne adduct. Final purification by washing residue with solvents afforded 10A (226 mg, 99%) as a white solid. LC-MS: [M+2H⁺]²⁺=769.

Compound 6A was prepared from 6 (90 mg, 0.061 mmol, 1 eq) and compound B (20 mg, 0.064 mmol, 1.05 eq) by a general procedure as described for peptido alkyne adduct. Final purification by washing residue with solvents afforded 6A (61.7 mg, 61.4%) as a white solid. LC-MS: [M+2H⁺]²⁺=825.

Compound 12A was prepared from 12 (100 mg, 0.068 mmol, 1 eq) and compound B (21.4 mg, 0.068 mmol, 1 eq) by a general procedure as described for peptido alkyne adduct. Final purification by washing residue with solvents afforded 12A (108.8 mg, 97.1%) as a white solid. LC-MS: [M+2H⁺]²⁺=825.

Compound 13A was prepared from 13 (90 mg, 0.064 mmol, 1 eq) and compound B (20.2 mg, 0.064 mmol, 1 eq) by a general procedure as described for peptido alkyne adduct. Final purification by washing residue with solvents afforded 13A (66.2 mg, 65.6%) as a white solid. LC-MS: [M+2H⁺]²⁺=790.

Compound 15A was prepared from Degarelix (20 mg, 0.012 mmol, 1 eq) and compound B (10 mg, 0.032 mmol, 2.5 eq) by a general procedure as described for peptido alkyne adduct. Final purification by washing residue with solvents afforded 15A (21.7 mg, 99%) as a white solid. LC-MS: [M+2H⁺]²⁺=905.

Compound 32 was prepared from 2 (10 mg, 0.007 mmol, 1 eq) by a general procedure as described for peptido azide adduct. Final purification by washing residue with solvents afforded 32 (8.6 mg, 84.4%) as a white solid.

### Radioligand Binding Assay

An amount of 2 μg of a purified membrane with LHRHR, also known as GnRHR, was incubated with 0.18 nM [125I]-[D-Trp6]-LHRH and compounds of interest in the incubation buffer (50 mM HEPES, pH 7.4, 5 mM MgCl2, 1 mM CaCl2, and 0.2% BSA). The reaction mixtures were incubated for 1 h at 25° C. and then were transferred to a 96-well GF/B filter plate (Millipore Corp., Billerica, MA, USA), which were were terminated by manifold filtration and washed with a wash buffer (50 mM HEPES, pH 7.4, and 100 mM NaCl) for six times. The radioactivity bound to the filter was measured by a Topcount® system (PerkinElmer Inc., Waltham, MA, USA). IC₅₀ values were determined by the concentration of a compound required to inhibit 50% of the specific binding of [125I]-[D-Trp6]-LHRH and calculated by nonlinear regression (GraphPad Software, San Diego, CA, USA). The design of these drug conjugates is to actively targeting LHRH-receptor at tumor site and facilitate drug conjugate accumulation in situ. Therefore, selective and strong binding of the synthetic peptide ligand with the receptor is important to allow enrichment of these conjugates, followed by release of the drug payload in the tumor microenvironment.

The results are shown in Table 1. Compounds of the invention have a binding as low as 0.17 nM.

TABLE 1

| | Binding Assay | | |
|---|---|---|---|
| Compound | (nM) | Mass | Purity (%) |
| 14 | +++ | [M + 2H⁺]²⁺ = 1149 | 96 |
| 15 | +++ | [M + 2H⁺]²⁺ = 1356 | 95.2 |
| 1 | + | [M + 2H⁺]²⁺ = 699 | 98 |
| 2 | ++++ | [M + 2H⁺]²⁺ = 702 | 97 |
| 3 | ++ | [M + 2H⁺]²⁺ = 724 | 97 |
| 4 | + | [M + 2H⁺]²⁺ = 769 | 96 |
| 5 | + | [M + 2H⁺]²⁺ = 793 | 96 |
| 7 | + | [M + 2H⁺]²⁺ = 1236 | 96 |
| 8 | ++++ | [M + 2H⁺]²⁺ = 1114 | 97 |
| 9 | +++ | [M + 2H⁺]²⁺ = 1529 | 96 |
| 10 | +++ | [M + 2H⁺]²⁺ = 1228 | 96 |
| 11 | ++ | [M + 2H⁺²⁺ = 1449 | 95 |
| 14 | ++ | [M + 2H⁺]²⁺ = 737 | 97 |
| 16 | ++++ | [M + 2H⁺]²⁺ = 1414 | 95 |
| 18 | +++ | [M + 2H⁺]²⁺ = 1268 | 96 |
| 25 | ++++ | [M + 2H⁺]²⁺ = 1289 | 95 |
| 26 | +++ | [M + 2H⁺]²⁺ = 1324 | 95 |
| 29 | ++ | [M + 2H⁺]²⁺ = 1377 | 97 |
| 30 | ++++ | [M + 2H⁺]²⁺ = 1342 | 95 |
| 17 | ++++ | [M + 2H⁺]²⁺ = 1367 | 96 |
| 19 | +++ | [M + 2H⁺]²⁺ = 1456 | 96 |
| 20 | ++++ | [M + 2H⁺]²⁺ = 1173 | 95 |
| 21 | ++++ | [M + 2H⁺]²⁺ = 1414 | 96 |
| 22 | ++++ | [M + 2H⁺]²⁺ = 1264 | 95 |
| 12 | ++++ | [M + 2H⁺]²⁺ = 737 | 97 |
| 13 | ++ | [M + 2H⁺]²⁺ = 702 | 99 |
| 23 | ++++ | [M + 2H⁺]²⁺ = 1149 | 96 |
| 24 | +++ | [M + 2H⁺]²⁺ = 1114 | 96 |
| 27 | + | [M + 2H⁺]²⁺ = 1414 | 95 |
| 28 | +++ | [M + 2H⁺]²⁺ = 1449 | 95 |
| 31 | ++++ | [M + 2Na⁺]²⁺ = 1155 | 95 |

Note that + indicates an IC₅₀ of greater than 100 nM, ++: 10 nM to 100 nM, +++: 1 nM to 10 nM, and ++++: 0.1 nM to 1 nM.

### Pharmacokinetic Properties

Pharmacokinetic properties of compounds of the invention were measured following the procedures described in Liu et al., Bioconjugate Chem. 2017, 28, 7, 1878-1892.

To address the longevity of the intact conjugate during in vivo systemic circulation, single intravenous dose pharmacokinetic studies showed a reduction of clearance (CL) rate and volume distribution Vss of these conjugates, suggesting increased systemic stability and improved distributions. In particular, conjugates 14, 8, and 9 exhibited extensive AUC coverage. Further, results from conjugates 11 and 16 demonstrate conjugates of this invention advantageously achieve a similar exposure using different dosing frequencies.

TABLE 2
In vivo pharmacokinetic profile of conjugates

| | Mouse IV (dose: 5 mg/kg) | | |
|---|---|---|---|
| Compound | CL (ml/min/kg) | Vₛₛ (I/kg) | AUC(0-24 hr) (ng/ml * hr) |
| 14 | 0.9 | 0.2 | 88307 |
| 8 | 1.6 | 0.25 | 55091 |
| 9 | 6.7 | 0.2 | 12453 |
| 11 | 4 | 0.1 | 20975 |
| 16 | 15.5 | 0.5 | 4859 |
| 27 | 6.4 | 0.1 | 15948 |

### In Vivo Anti-Tumor Activities

In vivo anti-tumor activity of Compound 16 was evaluated in ovarian OVCAR-3 tumor model. OVCAR-3 cells were suspended with RPMI 1640 medium (phenol red free) and Matrigel™ in 1:1 ratio, and implanted subcutaneously (1×10⁶ cells/flank) into the left flank of the immunodeficient (NOD/SCID) mice. The tumor volume in mm³ was calculated by the formula: Volume=(length×width\hat{ }2)/2. Tumor-bearing mice were randomized (n=8 per group) when the average tumor volume was approximately at 190 mm³ on day 49 after inoculation. Compound 16 was dissolved in DMA, cremorphor and 5% dextrose solution (1:2:27, v/v/v). Tumor-bearing mice were administered intravenously with vehicle control (5 ml/kg) and Compound 16 (1.5 or 2.5 mg/kg) once per week for 6 weeks on Day 1, 8, 15, 22, 29 and 36. Tumor size and body weight of each animal were measured twice weekly during the study period.

In vivo anti-tumor activity of Compound 16 was evaluated in ovarian A2780 tumor model. A2780 cells were suspended with RPMI 1640 medium (phenol red free) and Matrigel™ in 1:1 ratio, and implanted subcutaneously (1×10⁶ cells/flank) into the left flank of the immunodeficient nude mice. The tumor volume in mm³ was calculated by the formula: Volume=(length×width\hat{ }2)/2. Tumor-bearing mice were randomized (n=8 per group) when the average tumor volume was approximately at 390 mm³ on day 13 after inoculation. Compound 16 was dissolved in DMA, cremorphor and 5% dextrose solution (1:2:7, v/v/v). Tumor-bearing mice were administered intravenously with vehicle control (2.5 mL/kg) and Compound 16 (1.5 or 2 mg/kg) twice per week for two weeks on Day 1, 4, 8 and 11. Tumor size and body weight of each animal were measured twice weekly during the study period.

In vivo anti-tumor activity of Compound 16 was evaluated in triple-negative breast HCC1806 tumor model. HCC1806 cells were suspended with RPMI 1640 medium (phenol red free) and Matrigel™ in 1:1 ratio, and implanted subcutaneously (1×10⁶ cells/flank) into the left flank of the immunodeficient nude mice. The tumor volume in mm³ was calculated by the formula: Volume=(length×width\hat{ }2)/2. Tumor-bearing mice were randomized (n=8 per group) when the average tumor volume was approximately at 210 mm³ on day 10 after inoculation. Compound 16 was dissolved in DMA, cremorphor and 5% dextrose solution (1:2:27, v/v/v). Tumor-bearing mice were intravenously administered once weekly (qw) or bi-weekly (b2w) with multiple doses of vehicle control (5 mL/kg) and DBPR376 (2.5 mg/kg). Tumor size and body weight of each animal were measured twice weekly during the study period.

Compounds 8, 9, 11, 14, 16, 25, 26, and 27 were evaluated following the procedure described above. Each compound effectively inhibited in vivo the tumor growth of the anti-HCC1806 triple negative breast cancer at 1 mg/kg to 2.5 mg/kg when administered to the mice every other week, once a week, or twice a week. Surprisingly, Compounds 9 and 14 inhibited within 15 days 100% the growth (i.e., no cancer progression) of the anti-HCC1806 triple negative breast cancer at 1 mg/kg when administered to the mice twice a week; Compound 16 inhibited within 30 days 100% the growth of the anti-HCC1806 triple negative breast cancer at 1.5 mg/kg when administered to the mice twice a week; Compounds 25 and 26 inhibited within 30 days 100% the growth of the anti-HCC1806 triple negative breast cancer at 2 mg/kg when administered to the mice twice a week; Compounds 16 and 27 inhibited within 30 days or up to 100 days 100% the growth of the anti-HCC1806 triple negative breast cancer at 2.5 mg/kg when administered to the mice once a week; and Compound 16 inhibited within 60 days 100% the growth of the anti-HCC1806 triple negative breast cancer at 2.5 mg/kg when administered to the mice once a week.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

Further, from the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

What is claimed is:

1. A compound of formula (I): 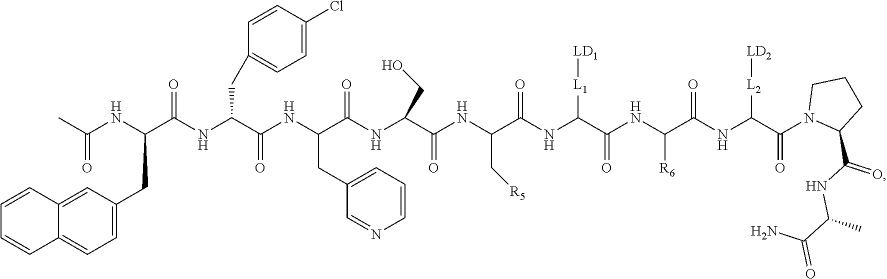
in which
each of L₁ and L₂, independently, is C₁-C₆ alkyl, aryl, or aralkyl;
LD₁ is —N(R₁)(R₂) or a first linker-drug moiety represented by -LK1-DG1, in which R₁ is H, R₂ is H or substituted carbonyl, or R₁ and R₂, together with the nitrogen atom they attached to, form —N₃, substituted or unsubstituted guanidino, 5- to 14-membered heteroaryl, or 5- to 14-membered heterocycloalkyl; LK1 is a first linker; and DG1 is a first drug moiety;
LD₂ is —N(R₃)(R₄) or a second linker-drug moiety represented by -LK2-DG2, in which R₃ is H, R₄ is substituted carbonyl, or R₃ and R₄, together with the nitrogen atom they attached to, form substituted or unsubstituted guanidino, 5- to 14-membered heteroaryl, or 5- to 14-membered heterocycloalkyl; LK2 is a second linker; and DG2 is a second drug moiety;
R₅ is aryl, C₁-C₆ alkyl, or a third linker-drug moiety represented by -LK3-DG3, in which LK3 is a third linker and DG3 is a third drug moiety; and
R₆ is C₁-C₆ alkyl.

2. The compound of claim 1, wherein each of LD₁ and LD₂, independently, is —NH₂, —N₃, —NHC(O)NH₂, —NHC(═NH)NH₂, or —N═C(NHCH₂CH₃)₂.

3. The compound of claim 2, wherein LD₂ is —NHC(O)NH₂, —NHC(═NH)NH₂, or —N═C(NHCH₂CH₃)₂.

4. The compound of claim 1, wherein one of LD₁ and LD₂ is —NHC(O)NH₂, —NHC(═NH)NH₂, or —N═C(NHCH₂CH₃)₂, and the other is the first or second linker-drug moiety.

5. The compound of claim 4, wherein one of LD₁ and LD₂ is selected from the group consisting of

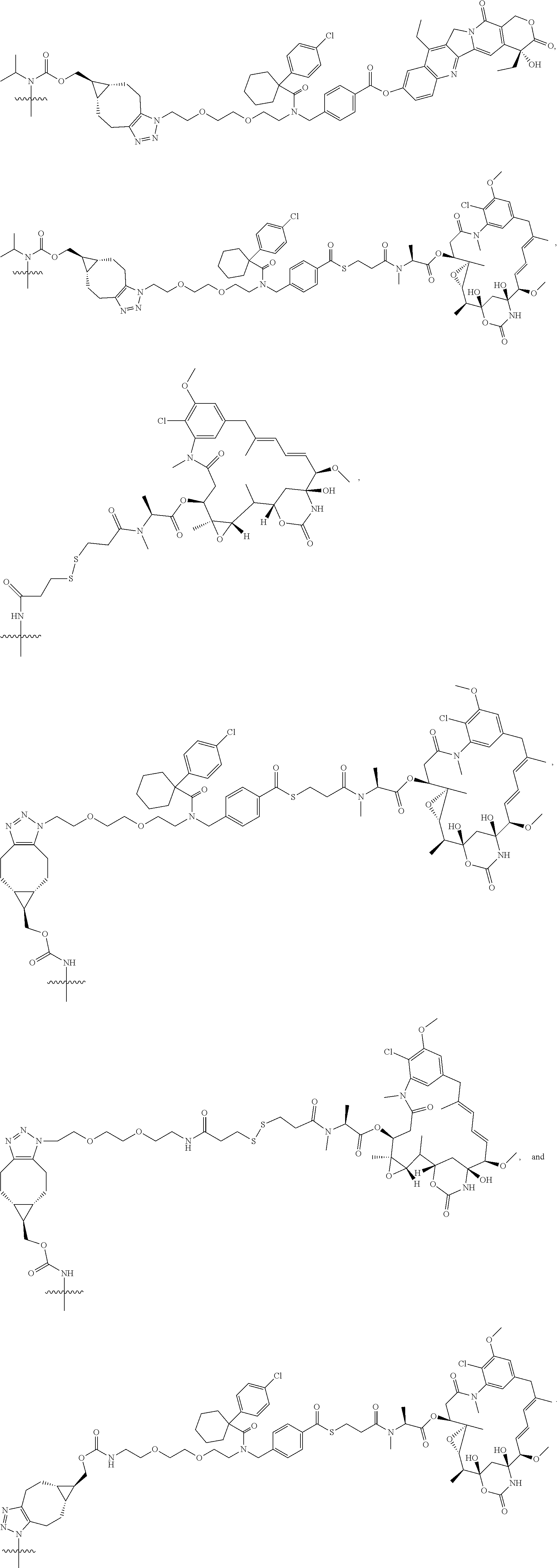

6. The compound of claim 1, wherein R₅ is substituted phenyl or substituted C₁-C₆ alkyl.

7. The compound of claim 6, wherein R₅ is aminopropyl, 4-hydroxyphenyl, or

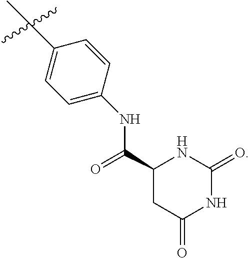 [SMILES: *c1ccc(NC(=O)[C@@H]2CC(=O)NC(=O)N2)cc1]

8. The compound of claim 1, wherein R₆ is isobutyl or aminobutyl.

9. The compound of claim 1, wherein L₁ is ethylene, propylene, or phenylene.

10. The compound of claim 1, wherein the compound is of formula (III): 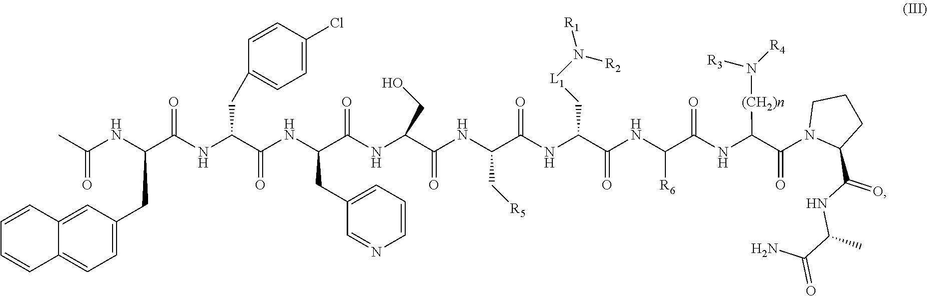
in which n is an integer from 1 to 6.

11. The compound of claim 10, wherein n is 3 or 4.

12. The compound of claim 1, wherein the compound is one of Compounds 1-32.

13. The compound of claim 1, wherein the compound is one of Compounds 8, 9, 11, 14, 16, and 25-28.

14. A pharmaceutical composition comprising a compound of claim 1 and a pharmaceutically acceptable carrier.

15. A method of treating cancer comprising administering an effective amount of a compound of claim 1 to a patient in need thereof.

16. The method of claim 15, wherein the cancer is colorectal cancer, renal cell carcinoma, lung cancer, pancreatic cancer, endometrial cancer, ovarian cancer, breast cancer, or prostate cancer.

17. The method of claim 15, wherein the cancer is ovarian cancer or breast cancer.
